(19) United States
(12) Patent Application Publication
(10) Pub. No.: US 2019/0183445 A1
(43) Pub. Date: Jun. 20, 2019

(54) DIAGNOSTIC IMAGING SYSTEM

(71) Applicant: SHIMADZU CORPORATION, Kyoto (JP)

(72) Inventors: Tomoharu OKUNO, Kyoto (JP); Shinsuke KANAZAWA, Kyoto (JP); Daisuke NOTOHARA, Kyoto (JP); Kazuhiro MORI, Kyoto (JP)

(73) Assignee: Shimadzu Corporation, Kyoto (JP)

(21) Appl. No.: 16/091,208

(22) PCT Filed: Feb. 20, 2017
(86) PCT No.: PCT/JP2017/006219

§ 371 Date: Oct. 4, 2018

(30) Foreign Application Priority Data
Apr. 5, 2016 (JP) PCT/JP2016/061170

(51) Int. Cl. A61B 6/00; A61B 6/12; A61B 10/00; A61B 10/04; A61B 90/90

(52) U.S. Cl. CPC A61B 6/5247; A61B 6/12; A61B 90/90; A61B 10/04; A61B 10/0096

(57) ABSTRACT

This diagnostic imaging system (100) is provided with an acquisition means (50) configured to acquire a diagnostic image (40) of a subject (T) and an association means (60) configured to associate the diagnostic image capable of identifying a collection position when a specimen sample is collected from the subject among diagnostic images acquired by the acquisition means with information (42) which identifies the specimen sample collected from the subject.

### TECHNICAL FIELD

The present invention relates to a diagnostic imaging system.

### BACKGROUND ART

Conventionally, it is known to perform diagnosis of diseases caused by a tumor, etc., in a body and organ by collecting a specimen sample, such as, e.g., blood and tissue piece, from the body of a subject (patient). As a collection method of a specimen sample, there are a blood collection, a biopsy with a collection needle, a tissue piece collection by a surgical operation, a collection using a collection device of a type to be introduced into a body, and the like. For example, in the case of using a collection device, a doctor feeds the collection device for collecting a specimen sample to a local part in a subject while confirming a fluoroscopic image of the subject by a radiation image diagnostic device to collect a specimen sample. The collected specimen is analyzed by a specimen analyzing device or pathologically examined by a microscope, etc., and a diagnosis is performed based on the analysis result and the examination result.

In a Non-Patent Document, it is disclosed to perform blood collection from veins at various parts of adrenal glands for diagnosis of primary aldosteronism by inserting a catheter to a collection position while confirming the X-ray fluoroscopic image of the subject by the radiographic image diagnostic apparatus in real time. The blood (specimen sample) collected by adrenal vein collection at each position is analyzed and the definitive diagnosis is performed based on the cortisol concentration as an analysis result, etc.,

When the definitive diagnosis is performed based on the analysis result and/or the examination result, a lesion is identified based on the collection position of the collected sample, and then whether or not the partial resection, etc., of the lesion is performed is determined. For this reason, it is necessary to strictly manage so that there is no mistake in the correspondence relation between the lesion analysis result and the blood collection position. In Non-Patent Document 1, it is disclosed that for the purpose of managing the correspondence relation between the collected lesion and the blood collection position, a label to which a blood collection number is written is attached to a blood collection tube, and at the same time the blood collection position is written in the clinical record together with the sketch of the adrenal vein. These tasks are carried out in cooperation with radiologists, internal physicians, and other related workers who perform the blood collection procedure.

### PRIOR ART

### Non-Patent Document

Non-Patent Document 1: Kohzoh Makita, “Adrenal Venous Collecting for Primary Aldosteronism—Tips and Tricks for Successful AVS Procedure”, Journal of the Japan Interventional Radiology Society, 2013, Vol. 28, No. 2, p. 204-210″

### SUMMARY OF THE INVENTION

### Problems to be Solved by the Invention

As described in Non-Patent Document 1, conventionally, for the purpose of preventing false recognition of the correspondence relation between the analysis result of the specimen sample and the collection position of the specimen sample, it is necessary that a plurality of doctors is present at an examination to confirm or a doctor in charge makes an effort such as matching the blood collection position with the analysis result based on the sketch. For this reason, the burden on doctors and workers involved in a local diagnosis is large. Thus, it is desired to reduce the administrative burden of the analysis result and the collection position of the specimen sample when performing the local diagnosis.

The present invention has been made to solve the aforementioned problems, and one of the objects of the present invention is to provide a diagnostic imaging system capable of reducing a management burden of an analysis result and a collection position of a specimen sample when conducting a diagnosis by a specimen sample collected from a subject.

### Means for Solving the Problems

In order to attain the aforementioned object, a diagnostic imaging system according to one aspect of the present invention includes: an acquisition means configured to acquire a diagnostic image of a subject; and an association means configured to associate a diagnostic image capable of identifying a collection position when a specimen sample is collected from the subject among the diagnostic image acquired by the acquisition means with information which identifies the specimen sample collected from the subject.

In the diagnostic imaging system according to the first aspect of the present invention, as described above, it is provided with an association means configured to associate a diagnostic image capable of identifying a collection position when a specimen sample is collected from the subject with information which identifies a specimen sample collected from the subject. With this, it becomes possible for a doctor or the like to identify the collection position of the specimen sample from the diagnostic image obtained when the specimen sample (for example, the tissue piece) is collected from the subject. By associating the diagnostic image at the time of collecting the specimen sample with the information which identifies the specimen sample collected from the subject, for example, when a doctor identifies the collection position of the specimen sample from the diagnostic image, it is possible to easily identify the specimen sample associated with the identified collection position. When the analysis result of the specimen sample is obtained, with the diagnostic image associated with the information which identifies the specimen sample, it becomes possible to associate the collection position of the specimen sample with the analysis result of the specimen sample. As a result, without creating a sketch at the time of collecting the specimen sample or associating the collection position and the analysis result of the specimen sample based on the sketch, the corresponding relation between the collected specimen sample and the collection position (showing the diagnostic image) can be managed. As described above, according to the present invention, it becomes possible to reduce the management burden of the analysis result and the collection position of the specimen sample when conducting a diagnosis by a specimen sample collected from the subject.

In the diagnostic imaging system according to the one aspect of the present invention, it is preferable that the diagnostic image include at least one of an X-ray image, a CT image, an MRI image, an ultrasonic image, a nuclear medicine image, and an optical image. With this configuration, it is possible to associate the information which identifies the specimen sample with the various diagnostic images suitable for diagnoses of diseases to thereby associate the specimen sample with the collection position. As a result, it is possible to provide a versatile diagnostic imaging system capable of associating various diagnostic images with specimen samples.

In the diagnostic imaging system according to the one aspect of the present invention, it is preferable that the diagnostic image include at least one of a two-dimensional image and a three-dimensional image. With this configuration, it becomes possible to associate a two-dimensional image and a three-dimensional image with the information which identifies the specimen sample. As a result, when a doctor identifies the collection position of the specimen sample from the diagnostic image, depending on the collection part or position, an appropriate diagnostic image that makes it easier to identify the collection position can be associated with the specimen sample.

In the diagnostic imaging system according to the one aspect of the present invention, it is preferable that the diagnostic image include at least one of a still image and a moving image. With this configuration, it becomes possible to associate a still image or a moving image with the information which identifies a specimen sample. For example, by using a moving image format diagnostic image showing the situation of the sample collection, it becomes possible to utilize an appropriate diagnostic image. For example, it becomes possible for a doctor to easily identify the collection position of the specimen sample from the diagnostic image.

In the diagnostic imaging system according to the one aspect of the present invention, it is preferable that the diagnostic image capable of identifying the collection position include an image capable of identifying the collection position of the specimen by a sample collection device arranged at the collection position or near the collection position. With this configuration, when collecting body tissues that are difficult to recognize from a diagnostic image, blood of a local part, etc., it is possible to easily identify the collection position from the position of the specimen collection device for collection.

In this case, it is preferable that the sample collection device include a collection tool configured to be introduced in the subject to collect a specimen sample in the subject. Here, the collection tool is a concept including a puncture needle, an endoscope, a capsule endoscope, a catheter, and the like. With this configuration, since a diagnostic image showing a collection tool introduced up to a collection position of a specimen sample in a subject can be obtained, the collection position of the specimen sample can be easily identified.

In the diagnostic imaging system according to the one aspect of the present invention, it is preferable that the diagnostic image capable of identifying the collection position include an image capable of identifying the collection position by at least one of a marker introduced in the subject and an indwelling object in the subject. Here, the indwelling object includes medical equipment indwelled in a body, such as, e.g., a stent, a coil, and an artificial valve. With this configuration, unlike internal organs, the collection position of the specimen sample can be easily identified by the diagnostic image showing the marker or the indwelling object which can easily obtain high visibility on an X-ray image or other images.

In the diagnostic imaging system according to the one aspect of the present invention, it is preferable that information which identifies the specimen sample collected from the subject include identification information assigned for each specimen sample at the time of collection. With this configuration, by assigning unique identification information for each specimen sample when the specimen sample is collected, it becomes possible to easily and assuredly associate the collection position of the specimen sample with the diagnostic image capable of identifying the collection position of the specimen sample.

In the diagnostic imaging system according to the one aspect of the present invention, it is preferable that information which identifies the specimen sample collected from the subject include identification information to be attached to a specimen container for accommodating a collected specimen sample. With this configuration, when collecting a specimen sample, simply entering the identification information to be attached to the specimen container makes it possible to easily associate the diagnostic image with the identification information.

In the diagnostic imaging system according to the one aspect of the present invention, it is preferable that information which identifies the specimen sample collected from the subject include identification information received from at least one of a specimen analyzing device for analyzing the specimen sample and a server recording an analysis result of the specimen sample. With this configuration, the identification information can be easily obtained from a server or a specimen analyzing device, and automatic association can be performed. As a result, the convenience of the diagnostic imaging system can be improved.

In the diagnostic imaging system according to the one aspect of the present invention, it is preferable that the association means further associate information which identifies the subject with each of a plurality of diagnostic images associated with information which identifies the specimen sample collected from the subject. With this configuration, when the association between the collected specimen sample and the diagnostic image which identifies the collection position is performed multiple times on the same subject, each diagnostic image (and specimen sample) can be managed collectively by the information which identifies the subject. This makes it easy to grasp the multiple examination results at time intervals with respect to the same subject in chronological order, which enables an easy follow-up of the patient (subject).

In the diagnostic imaging system according to the one aspect of the present invention, it is preferable that the association means further associate the information which identifies the collection position of the specimen sample in the diagnostic image with the diagnostic image when the specimen sample is collected. With this configuration, not only the collection position of the specimen sample can be identified on the diagnostic image, but also the collection position can be grasped by the information which identifies the collection position associated with the diagnostic image. For this reason, it becomes possible to effectively reduce the management burden of the analysis result and the collection position of the specimen sample.

In the diagnostic imaging system according to the one aspect of the present invention, it is preferable that the association means further associate the information which identifies the collection position of the specimen sample in the diagnostic image with the information which identifies the specimen sample collected from the subject. With this configuration, not only the collection position of the specimen sample can be identified on the diagnostic image, but also the collection position can be grasped by the information which identifies the collection position associated with the information which identifies the specimen sample. For this reason, it becomes possible to effectively reduce the management burden of the analysis result and the collection position of the specimen sample.

In the configuration in which the information which identifies the collection position of the specimen sample is associated with the diagnostic image or the configuration in which the information which identifies the collection position of the specimen sample is associated with the information which identifies the specimen sample, it is preferable that the information which identifies the collection position include a position coordinate of the collection position in the diagnostic image. With this configuration, it is possible to clearly and assuredly grasp the collection position in the diagnostic image by the position coordinate.

In the configuration in which the information which identifies the collection position of the specimen sample is associated with the diagnostic image or the configuration in which the specimen sample is associated with the information identifying the specimen sample, it is preferable that the information which identifies the collection position include a relative position of the collection position with respect to a feature point reflected in the diagnostic image. Here, the feature point includes an anatomical structure, such as, e.g., a blood vessel and a bone in a diagnostic image, and medical equipment, such as, e.g., a marker and a stent in a body. With this configuration, it is possible to easily grasp the collection position in the diagnostic image by the relative position of the collection position with respect to the feature point in the subject. Further, the feature point in the subject is used as a reference for a collection position. Therefore, for example, when a doctor compares multiple diagnostic images, even when the collection position is shifted between diagnostic images due to the subject's own movements or the like, as long as the feature point moves with the collection position, the collection position (relative position) with respect to the feature point does not shift, which enables accurate grasping of the collection position.

In the configuration in which the information which identifies the collection position of the specimen sample is associated with the diagnostic image or the configuration in which the specimen sample is associated with the information identifying the specimen sample, it is preferable that the information which identifies the collection position include an anatomical name of a part to which the collection position of specimen sample belongs. With this configuration, the anatomical name makes it possible to intuitively and promptly understand the collection position when a doctor, etc., refers to the diagnostic image. For this reason, it becomes easy to grasp the collection position and improve the convenience of the diagnostic imaging system.

In the diagnostic imaging system according to the one aspect of the present invention, it is preferable that the association means further associate an analysis result of the specimen sample with information which identifies the specimen sample collected from the subject. With such a configuration, it becomes possible to collectively manage the diagnostic image capable of identifying the collection position and the analysis result of the specimen sample collected from the collection position. For this reason, it becomes possible to more effectively reduce the management burden of the analysis result and the collection position of the specimen sample.

In this case, it is preferable that the analysis result of the specimen sample include a pathological diagnosis result for the specimen sample. With this configuration, when the presence or absence of a lesion or the type of a lesion is identified by the pathological diagnosis result, it becomes possible to directly grasp the lesion part (collection position of the specimen sample) from the diagnostic image. As a result, it is possible to facilitate grasping of the lesion part and to improve the convenience of the diagnostic imaging system.

In the configuration in which the analysis result of the specimen sample is associated with the information which identifies the specimen sample collected from the subject, it is preferable that the analysis result of the specimen sample include a component analysis result for the specimen sample. With this configuration, even when a lesion or the like is collected from a plurality of locations around the examination target part, it becomes possible to manage the component analysis result and the collection position of each specimen sample in an associated manner. This enables effective reduction of the management burden of the analysis result and the collection position.

A diagnostic imaging system according to the second aspect of the present invention includes: an acquisition means configured to acquire a diagnostic image capable of identifying a collection position of a specimen sample for each of a plurality of different collection positions; and an image synthesizing means configured to synthesize a plurality of diagnostic images to generate a synthesized image.

Here, in cases where specimen samples are collected from a plurality of different collection positions, at the time of diagnosis, in some cases, it becomes difficult to grasp where each collection position is positioned in the examination target part (organ, etc.). For example, in the case of acquiring a diagnostic image magnified to a high magnification so that the collection position can be clearly identified, in some cases, it becomes necessary for a doctor to distinguish each image by comparing them at the time of diagnosis, which increases the burden of the diagnostic work. Also, when explaining the diagnostic result to a patient, etc., since it is troublesome to explain each individual diagnostic image one by one, the doctor is sometimes required to perform the editing work so that each diagnostic image can be listed, which also increases the burden of the diagnostic work. Therefore, it is desired to make the doctor's diagnostic work more efficient by using diagnostic images.

Under the circumstance, in the diagnostic imaging system according to the second aspect of the present invention, as described about, an image synthesizing means configured to synthesize a plurality of diagnostic images to generate a synthesized image is provided. Thus, it is possible to comprehensively grasp a plurality of collection positions by the synthesized image obtained by synthesizing a plurality of diagnostic images capable of identifying each collection position. As a result, by referring to the synthesized image at the time of the diagnosis, the doctor can easily grasp each of the plurality of collection positions. When explaining the diagnosis result, it is unnecessary to present individual diagnostic images one by one to a patient or to edit each diagnostic image so that the images are listed. As a result, it becomes possible to make the doctor's diagnosis work and the explanation work to the patient using the diagnostic images more efficient. Further, since a plurality of collected positions can be collectively grasped by the synthesized image, it is possible to reduce the management burden of the analysis result and the collection position of specimen sample when conducting a diagnosis by the specimen sample collected from the subject.

In the diagnostic imaging system according to the second aspect of the present invention, it is preferable that the image synthesizing means collect images of regions including the collection position in each of the diagnostic images to generate a single synthesized image. With this configuration, it becomes possible to grasp each collection position collectively in a single synthesized image. Thus, it is possible to further facilitate grasping of each collection position by the diagnostic image at the time of diagnosis or explanation to a patient.

In the diagnostic imaging system according to the second aspect of the present invention, it is preferable that the image synthesizing means align an image of a region including the collection position in another diagnostic image with any of the diagnostic images and superimposes them to generate the synthesized image. With this configuration, based on the diagnostic image captured the entire examination target part (such as organ), a diagnostic image showing the detail of the individual collection position can be placed at the collection position in the base diagnostic image and superimposed. As a result, the synthesized image makes it possible to grasp the overall image of the examination target part and the arrangement and state of individual collection positions in the overall image at a glance.

In the diagnostic imaging system according to the second aspect of the present invention, it is preferable that the image synthesizing means generate the synthesized image to be displayed visually distinguishably by making display colors of the plurality of collection positions different from each other. With this configuration, since it becomes possible to distinguish a plurality of collection positions by color as well as position, it becomes possible to easily identify each collection position at a glance in a synthesized image. As a result, the doctor's diagnostic work using diagnostic images can be made more efficient.

### Effects of the Invention

According to the present invention, as described about, it becomes possible to reduce the management burden of the analysis result and the collection position of the specimen sample when conducting a diagnosis by a specimen sample collected from a subject.

### BRIEF DESCRIPTION OF THE DRAWINGS

FIG. 1 is a schematic diagram showing the overall configuration of a diagnostic imaging system according to a first embodiment.

FIG. 2 is a schematic diagram showing a configuration example of the diagnostic imaging system.

FIG. 3 is a diagram (A) to (E) showing images of various diagnostic images.

FIG. 4 is a diagram (A) showing a marker and diagrams (B) and (C) each showing an indwelling object.

FIG. 5 is a bock diagram showing the overall configuration of a diagnostic imaging system according to a second embodiment.

FIG. 6 is a block diagram for explaining a configuration example of an X-ray imaging apparatus.

FIG. 7 is a block diagram for explaining a configuration example of a specimen analyzing device.

FIG. 8 is a diagram for explaining an example of an X-ray image capable of identifying a collection position of a specimen sample in a subject.

FIG. 9 is a conceptual diagram for explaining an association between a collection number and an X-ray image and analysis result.

FIG. 10 is a diagram for explaining an example of image connection data.

FIG. 11 is a flowchart for explaining associating processioning according to a second embodiment.

FIG. 12 is a block diagram showing an overall configuration of a diagnostic imaging system according to a third embodiment.

FIG. 13 is a conceptual diagram for explaining an association between the time information and the X-ray image and the analysis result.

FIG. 14 is a flowchart for explaining the associating processioning according to the third embodiment.

FIG. 15 is a diagram for explaining a specimen collection button of the diagnostic imaging system according to a fourth embodiment.

FIG. 16 is a flowchart for explaining the associating processing according to the fourth embodiment.

FIG. 17 is a block diagram showing an overall configuration of a diagnostic imaging system according to a fifth embodiment.

FIG. 18 is a conceptual diagram for explaining the association between the identification information and the X-ray image and the analysis result.

FIG. 19 is a flowchart for explaining the associating processioning according to the fifth embodiment.

FIG. 20 is a schematic diagram for explaining the association of the subject information according to a sixth embodiment.

FIG. 21 is a diagram for explaining the function of the subject information.

FIG. 22 is a diagram for explaining the association of the collection position information according to a seventh embodiment.

FIG. 23 is a schematic diagram showing the overall configuration of the diagnostic imaging system according to an eighth embodiment.

FIG. 24 is a schematic diagram showing a first example of a synthesized image.

FIG. 25 is a schematic diagram showing a second example of a synthesized image.

FIG. 26 is a schematic diagram showing a third example of a synthesized image.

### EMBODIMENTS FOR CARRYING OUT THE INVENTION

Hereinafter, some embodiments of the present invention will be described with reference to the drawings.

### First Embodiment

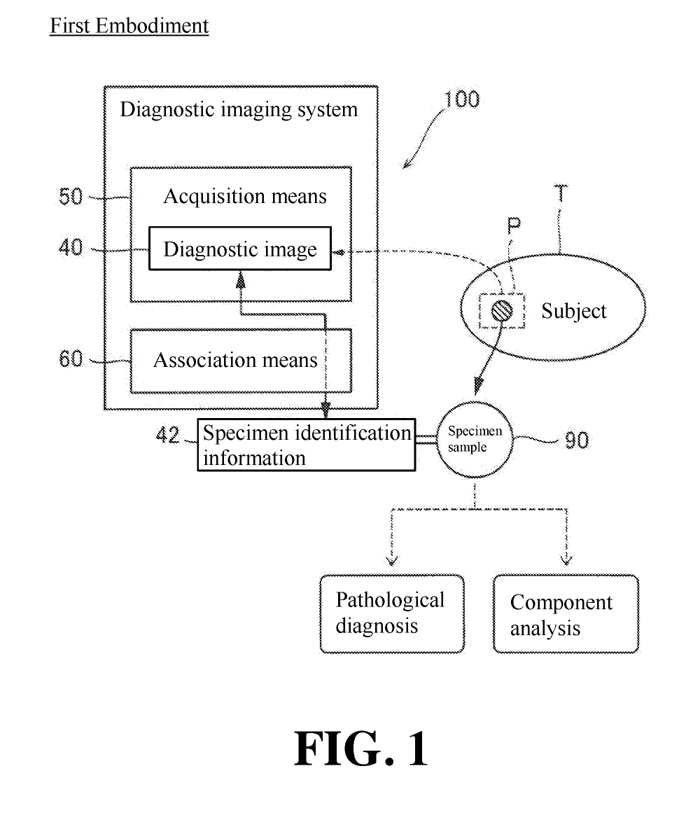

With reference to FIG. 1 to FIG. 4, the configuration of the diagnostic imaging system 100 according to a first embodiment of the present invention will be described.

The diagnostic imaging system 100 is a system configured to associate a diagnostic image 40 capable of identifying a collection position P when a specimen sample 90 is collected from a subject T with the information which identifies a specimen sample 90 (hereinafter referred to as “specimen identification information 42”). The specimen identification information 42 is information assigned to the specimen sample 90 collected from the subject T and capable of identifying the specimen sample 90. That is, the diagnostic imaging system 100 is configured to associate the specimen sample 90 collected from the subject T with the diagnostic image 40 indicating the collection position P of the specimen sample 90 by the specimen identification information 42.

The subject T is an object to which diagnosis of disease is performed, and the specimen sample 90 for diagnosis is collected from a subject T by a doctor, etc. The subject T includes humans and other animals.

The specimen sample 90 includes a general biological sample collected from a subject T, and is not particularly limited. The specimen sample 90 is, for example, a part or all of a body fluid, such as, e.g., blood or tissue fluid, and organs, such as, e.g., internal organs and bones.

Collection of the specimen sample 90 is performed by an appropriate method according to a collection target and a collection part. In cases where the specimen sample 90 is blood or tissue fluid, for example, such collection is performed by a method of collecting blood from a body of a subject T using a syringe equipped with a blood collection needle, a method of introducing a catheter for collecting blood (tissue fluid) into a body and collecting blood from a body of a subject T, or the like. In cases where the specimen sample 90 is a body tissue, such as, e.g., a part of an organ, collection is performed by a method of collecting a tissue of a collection part from an outside by performing a surgical operation, a method of collecting a tissue of a collection part from an inside by introducing a collection device into the body using an endoscope or a catheter, etc. The collected specimen sample 90 is subjected to an analysis, and an analysis result is generated. The analysis result of the specimen sample 90 includes, for example, a component analysis result with respect to a specimen sample 90 using a specimen analyzing device or a manual method. Further, the analysis result of the specimen sample 90 includes, for example, a pathological diagnosis result with respect to the specimen sample 90 using a microscope or the like.

In the case of performing a definitive diagnosis based on a component analysis result and a pathological diagnosis result, it is important to identify the lesion. The collection position P of the specimen sample 90 is important information for identifying the lesion in conjunction with the component analysis result of the specimen sample 90 and the pathological diagnosis result to prevent the mix-up of the specimen sample 90.

Therefore, in this embodiment, the diagnostic imaging system 100 is provided with an acquisition means 50 for acquiring the diagnostic image 40 of the subject T and an association means 60 for associating the diagnostic image 40 capable of identifying the collection position P with the specimen identification information 42.

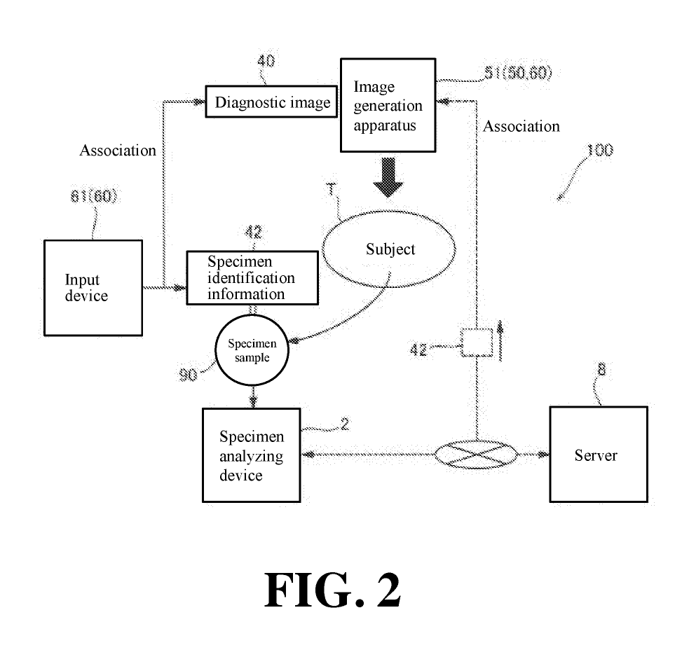

The acquisition means 50 acquires a diagnostic image 40 of a subject T generated by, for example, an image generation apparatus 51 (see FIG. 2). As a method of obtaining the diagnostic image 40, image data may be received by a wired or wireless transmission medium (network), or image data may be read from a portable recording medium in which the diagnostic image 40 is recorded. In the case of acquiring the data of the diagnostic image 40, the acquisition means 50 includes a computer capable of performing data communication and data reading.

The acquisition means 50 may acquire the diagnostic image 40 by, for example, generating the diagnostic image 40 of the subject T. That is, as shown in FIG. 2, the acquisition means 50 may include an image generation apparatus 51 for generating the diagnostic image 40 of the subject T.

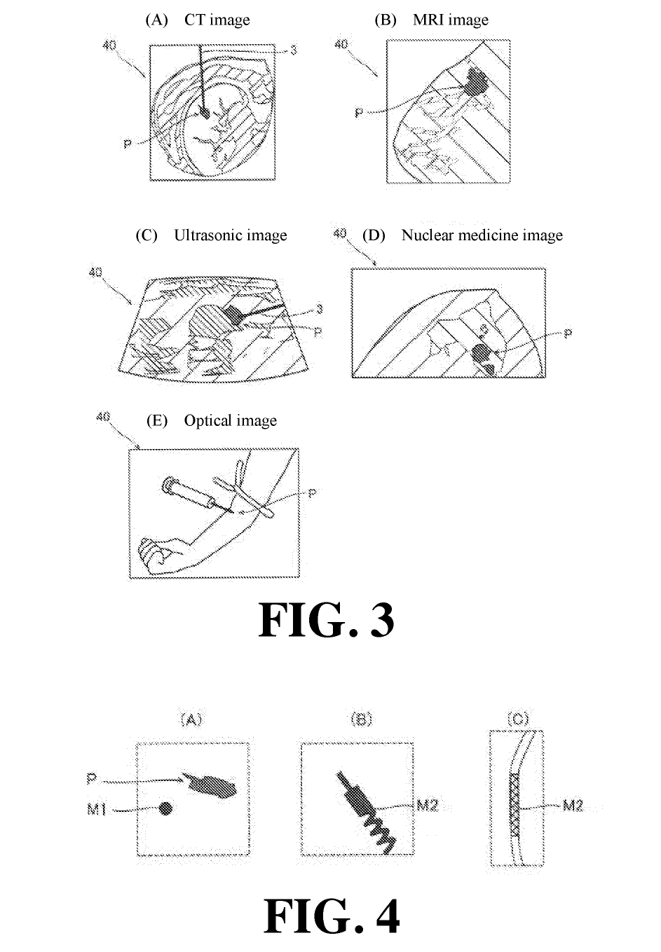

The diagnostic image 40 includes at least any one of an X-ray image (see FIG. 8), a CT image (see FIG. 3(A)), an MRI image (see FIG. 3(B)), an ultrasonic image (see FIG. 3(C)), the nuclear medicine image (see FIG. 3(D)), and the optical image (see FIG. 3(E)).

An X-ray image is an image (transmission image) of a subject T captured using radiation that passes through the subject T. A CT image is a cross-sectional image (tomographic image) in a subject T configured by subjecting the radiation image obtained by scanning the subject T to arithmetic processing An MRI image is a cross-sectional image in a subject T configured by subjecting a magnetic signal obtained using a nuclear magnetic resonance phenomenon to arithmetic processing. An ultrasonic image is an image configured by subjecting a reflected signal of an ultrasonic wave given in the subject T to imaging processing. A nuclear medicine image is an image showing distribution of a radioactive material configured by subjection a radiation signal released from a radioactive material administered in a subject T to arithmetic processing. The nuclear medicine image is, for example, a PET (positron emission tomography) image or a SPECT (single photon emission computed tomography) image. An optical image is an image using light (mainly visible light, but it can be infrared light) other than radiation, and shows an appearance of a subject T. The optical image may include, for example, an image obtained by imaging a blood collection position at the time of the blood collection and an image obtained by imaging the collection position P of the specimen sample 90 in a state in which a part of a body is exposed by a surgical operation.

Further, the diagnostic image 40 includes at least one of a two-dimensional image and a three-dimensional image. All of the above-mentioned X-ray image, a CT image, an MRI image, a ultrasonic image, a nuclear medicine image, and an optical image can be each generated as a two-dimensional image. Further, the CT image, the MRI image, and the nuclear medicine image can be each generated as a three dimensional image. Further, the diagnostic image 40 includes at least one of a still image and a moving image. That is, the diagnostic image 40 is not limited to a still image but may be in a moving image format which continuously images the time change of the imaging target.

Returning to FIG. 1, the association means 60 has a function of associating the diagnostic image 40 capable of identifying the collection position P when the specimen sample 90 is collected from the subject T among diagnostic images 40 acquired by the acquisition means 50 with the specimen identification information 42.

Typically, the diagnostic image 40 capable of identifying the collection position P is obtained by imaging (capturing the image of) the region including the collection position P in a viewable manner before or after the specimen sample 90 identified by the specimen identification information 42 is collected. Further, when the specimen sample 90 is collected from the subject T, specimen identification information 42 is assigned to the collected specimen sample 90 and managed.

The diagnostic image 40 capable of identifying the collection position P is image data generated separately from the specimen identification information 42 of the specimen sample 90 collected from the collection position P. Therefore, the data itself of the diagnostic image 40 is irrelevant to the specimen identification information 42. Therefore, the association means 60 performs associating processing, such as, e.g., recording the specimen identification information 42 assigned to the specimen sample 90 in the image file of the diagnostic image 40 capable of identifying the collection position P. As a result of the associating processing, it becomes possible to manage the diagnostic image 40 indicating the specific collection position P and the analysis result with respect to the specimen sample 90 collected at the collection position P or the specimen sample 90 in a state of being linked via the specimen identification information 42.

The diagnostic image 40 capable of identifying the collection position P is an image capable of identifying the collection position P of the specimen sample 90 or the collection position P by the specimen collection device 3 arranged near the collection position P. The sample collection device 3 includes a collection tool configured to be introduced in the subject T to collect a specimen sample in the subject T. Specifically, the collection tool includes a puncture needle (see FIG. 3(A) and FIG. 3(C)), an endoscope, a capsule endoscope (not shown), and a catheter (see FIG. 8). The specimen collection device 3 may be a blood collecting tool, such as, e.g., a syringe (see FIG. 3(E)). In the case of identifying the collection position P by the specimen collection device 3, the diagnostic image 40 is obtained by imaging the specimen collection device 3 placed at the collection position P (or near the collection position P) together with the collection position P in order to collect specimen sample 90 at the time of specimen sample 90 collection.

Further, the diagnostic image 40 capable of identifying the collection position P is, as shown in FIG. 4, an image capable of identifying the collection position P by at least one of the marker M1 introduced in the subject T and the indwelling object M2 in the subject T. The marker M1 (see FIG. 4(A)) is an object formed by, for example, a substance low in permeability of radiation, and its shape may be a spherical shape, a coiled shape, or the like, but not limited thereto. The indwelling object M2 includes medical equipment indwelled in a body, such as, e.g., a coil (see FIG. 4(B)), a stent (see FIG. 4(C)), and an artificial valve (not shown). In the case of identifying the collection position P by the marker M1 or the indwelling object M2, the diagnostic image 40 is obtained by imaging the marker M1 and/or the indwelling object M2 together with the collection position P before or during collecting of the specimen sample 90.

The specimen identification information 42 to be associated with the diagnostic image 40 may be any information as long as it can associate the diagnostic image 40 with the specimen sample 90 on a one-to-one basis. The specimen identification information 42 may be, for example, identification information entered by a user, such as, e.g., a doctor and a medical staff. In the case of accepting a user's input, as shown in FIG. 2, the association means 60 may include an input device 61. At the time of collecting the specimen sample 90, the input device 61 accepts an input of the identification number of the collected specimen sample 90 and assigns it to the specimen sample 90. In this case, the association means 60 performs the association by also assigning the identification number (specimen identification information 42) accepted by the input device 61 also to the diagnostic image 40.

The specimen identification information 42 may be identification information to be automatically generated by a device. The specimen identification 42 includes, for example, the identification information received from at least one of a specimen analyzing device 2 for analyzing the specimen sample 90 and a server 8 in which the analysis result of the specimen sample 90 is recorded. In the configuration that receives the specimen identification information 42, the association means 60 may be a receiving side device common to the acquisition means 50.

Taking the configuration of FIG. 2 as an example, for example, the acquisition means 50 and the association means 60 may be a common image generation apparatus 51. Upon generation of the diagnostic image 40, the image generation apparatus 51 as the association means 60 receives the specimen identification information 42 assigned to the specimen sample 90 from the specimen analyzing device 2 or the server 8. The received specimen identification information 42 is assigned to the diagnostic image 40. The diagnostic imaging system 100 may be configured as described above.

### Effects of First Embodiment

In the first embodiment, the following effects can be obtained.

In the first embodiment, as described above, it is provided with an association means 60 configured to associate the diagnostic image 40 capable of identifying the collection position P at the time of collecting the specimen sample 90 from the subject T with the specimen identification information 42. With this, it becomes possible to identify the collection position P of the specimen sample 90 from the diagnostic image 40 obtained when the specimen sample 90 is collected from the subject T. By associating the diagnostic image 40 at the time of collecting the specimen sample 90 with the information 42, for example, when a doctor identifies the collection position P of the specimen sample 90 from the diagnostic image 40, it is possible to easily identify the specimen sample 90 associated with the identified collection position P. When the analysis result of the specimen sample 90 is obtained, with the diagnostic image 40 associated with the specimen identification information 42, it is possible to make the collection position P of the specimen sample 90 associated with the analysis result. As a result, without creating a sketch at the time of collecting the specimen sample 90, the corresponding relation between the collected specimen sample 90 and the collection position P (showing the diagnostic image 40) can be managed. As described above, according to the diagnostic imaging system 100 of the first embodiment, it becomes possible to reduce the management burden of the analysis result and the collection position P of the specimen sample 90 when conducting a diagnosis by a specimen sample 90 collected from the subject T.

Further, in the first embodiment, as described above, the diagnostic image 40 is an image including at least one of an X-ray image, a CT image, an MRI image, an ultrasonic image, a nuclear medicine image, and an optical image. As a result, it is possible to associate the specimen sample 90 with the collection position P by associating the specimen identification information 42 with various diagnostic images 40 suitable for diagnosis of diseases. As a result, it is possible to provide a versatile diagnostic imaging system 100 capable of associating various diagnostic images 40 with the specimen sample 90.

Further, in the first embodiment, as described above, the diagnostic image 40 is an image including at least one of a two-dimensional image and a three-dimensional image. With this, when a doctor identifies the collection position P of the specimen sample 90 from the diagnostic image 40, depending on the collection part and the position, it is possible to associate the appropriate two-dimensional or three-dimensional diagnostic image 40 suitable for more suitable for identifying the collection position P with the specimen sample 90.

Further, in the first embodiment, as described above, it is preferable that the diagnostic image is an image including at least one of a still image and a moving image. With this, for example, by using the moving image format diagnostic image 40 showing the situation of the sample collection, it becomes possible to utilize an appropriate diagnostic image 40. For example, it becomes possible for a doctor to easily identify the collection position P of the specimen sample 90 from the diagnostic image 40.

Further, in the first embodiment, as described above, the diagnostic image 40 capable of identifying the collection position P is an image capable of identifying the collection position P of the specimen sample 90 or the collection position P by the specimen collection device 3 arranged near the collection position P. With this configuration, when collecting a body tissue that is difficult to recognize from a diagnostic image 40, blood of a local part, etc., it is possible to easily identify the collection position P from the position of the specimen collection device 3.

Further, in the first embodiment, as described above, as the sample collection device 3, a collection tool configured to be introduced in the subject T to collect a specimen sample in the subject T is adopted. With this configuration, since the diagnostic image 40 showing the collection tool introduced up to the collection position P of the specimen sample 90 in the subject T can be obtained, the collection position P of the specimen sample 90 can be easily identified.

Further, in the first embodiment, as described above, the diagnostic image 40 capable of identifying the collection position P is an image capable of identifying the collection position P by at least one of the marker M1 introduced in the subject T and the indwelling object M2 in the subject T. With this, unlike internal organs, the collection position P of the specimen sample 90 can be easily identified by the diagnostic image 40 showing the marker M1 or the indwelling object M2 which can easily obtain high visibility on an X-ray image or other images.

### Second Embodiment

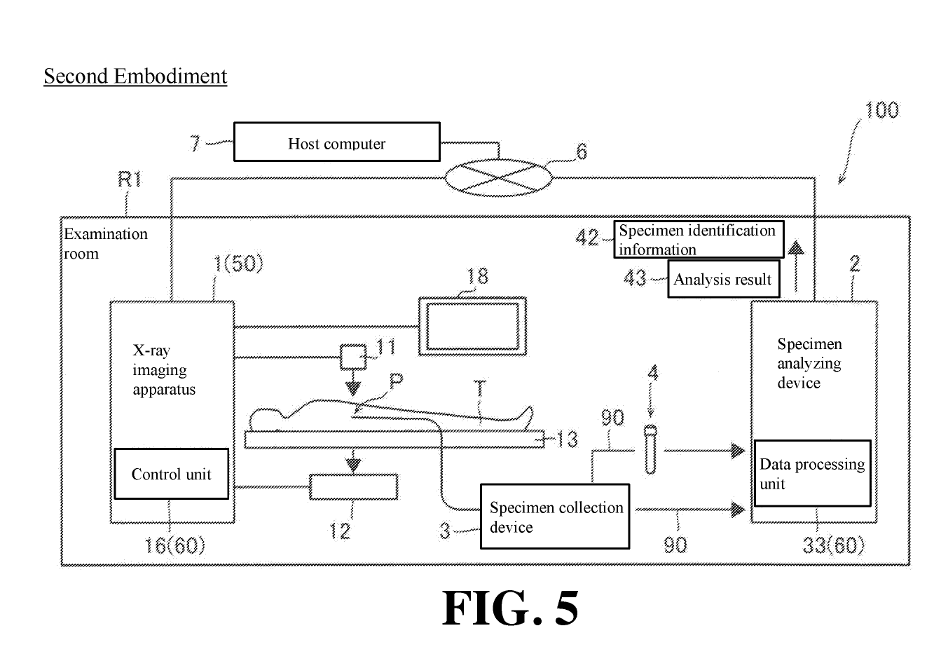

With reference to FIG. 5 to FIG. 10, the configuration of the diagnostic imaging system 100 according to the second embodiment of the present invention will be described. In the second embodiment, as a specific example of a diagnostic imaging system, a diagnostic imaging system 100 configured to perform X-ray imaging for collecting a specimen sample 90 and perform an analysis of a collected specimen sample 90 so as to perform the local diagnosis by collecting the specimen sample 90 in the subject T will be described.

(Diagnostic Imaging System)

Examples of a local diagnosis using the diagnostic imaging system 100 according to the second embodiment include adrenal vein collecting for diagnosis of primary aldosteronism, a selective arterial calcium injection test for diagnosis of insulinoma, and endoscopic biopsy performed by collecting an internal organs tissue piece using an endoscope. Hereinafter, in the case of showing a specific example of a local diagnosis, a case of adrenal vein collecting for a diagnosis of primary aldosteronism will be explained.

As shown in FIG. 5, the diagnostic imaging system 100 is provided with an X-ray imaging apparatus 1 for capturing an X-ray image 41 of a subject T and a specimen analyzing device 2 for analyzing a specimen sample 90 collected from the subject T. In the second embodiment, the X-ray imaging apparatus 1 and the specimen analyzing device 2 constituting the diagnostic imaging system 100 are installed in the examination room R1 of the medical institution, for example, and are operated by one or more operators such as doctors.

The diagnostic imaging system 100 captures an X-ray image from the outside of the subject T by the X-ray imaging apparatus 1 in order to collect a specimen sample 90 in the subject T. When collecting the specimen sample 90, the specimen collection device 3 is introduced inside the subject T. Using the captured X-ray image as a clue, a doctor in charge of the sample collection advances the specimen collection device 3 to the collection position P of the specimen sample 90 to collect the specimen sample 90.

For the adrenal vein collecting, a catheter is used as the specimen collection device 3.

The collected specimen sample 90 is taken into the specimen collection device 3 and transferred directly to the specimen analyzing device 2, or specially accommodated in a specimen container 4 for accommodating the specimen sample 90 and then the specimen container 4 is transferred to the specimen analyzing device 2. When the specimen analyzing device 2 is connected to the specimen collection device 3, the specimen analyzing device 2 is configured to capture the specimen sample 90 directly from the specimen collection device 3. In the case of using the specimen container 4, an operator such as a doctor sets the specimen container 4 to the specimen analyzing device 2, and the specimen analyzing device 2 accepts the specimen sample 90. The specimen container 4 is, for example, a blood collection tube. The specimen analyzing device 2 analyzes the acquired specimen sample 90.

While the specimen sample 90 is collected by the specimen collection device 3, the X-ray imaging apparatus 1 generates an X-ray image in a video format and displays it on a display unit 18. Further, the X-ray imaging apparatus 1 can record (save) the image of the arbitrary frame of the X-ray image in a video format as a still image at an arbitrary timing. In the second embodiment, an X-ray image 41 (see FIG. 8) capable of identifying the collection position P of the specimen sample 90 in the subject T is recorded in the still image format. The X-ray image 41 capable of identifying the collection position P may be recorded in the moving image format.

Specifically, the X-ray image 41 capable of identifying the collection position P of the specimen sample 90 is an image imaging the state in which the specimen collection device 3 is placed at the collection position P in the subject T. In the case of adrenal vein collecting, the tip end portion 3a (see FIG. 8) of the catheter is placed at the blood collection position of the adrenal vein to be collected out of the various adrenal veins and blood collecting is performed with the catheter indwelled. The X-ray image 41 is an image captured a state in which the tip end portion 3a of the catheter is placed at the blood collection position at the time of blood collecting. Looking at the recorded X-ray image 41, the actual blood collection position can be identified.

The association means 60 may be provided separately from the X-ray imaging apparatus 1 and the specimen analyzing device 2, but may be constituted by the X-ray imaging apparatus 1 or the specimen analyzing device 2. In other words, the X-ray imaging apparatus 1 or the specimen analyzing device 2 may be configured to function as the association means 60. In the second embodiment, the association means 60 is configured by the control unit 16 of the X-ray imaging apparatus 1 and the data processing unit 33 of the specimen analyzing device 2. The control unit 16 and the data processing unit 33 are examples of the “association means” recited in in claims.

In the second embodiment, the X-ray imaging apparatus 1 and the specimen analyzing device 2 are configured to be communicable with each other via a network 6 such as a LAN (Local Area Network). The X-ray imaging apparatus 1 and the specimen analyzing device 2 are configured to be able to transmit and receive the data of the analysis result 43 and the data of the specimen identification information 42 via the network 6, and to transmit and receive the control signal for exchanging data. The association means 60 acquires the analysis result 43 and specimen identification information 42 via the network 6 and associates with the recorded X-ray image 41. The association means 60 may be, for example, a host computer (server) 7 connected to each of the X-ray imaging apparatus 1 and the specimen analyzing device 2 via the network 6.

(X-Ray Imaging Apparatus)

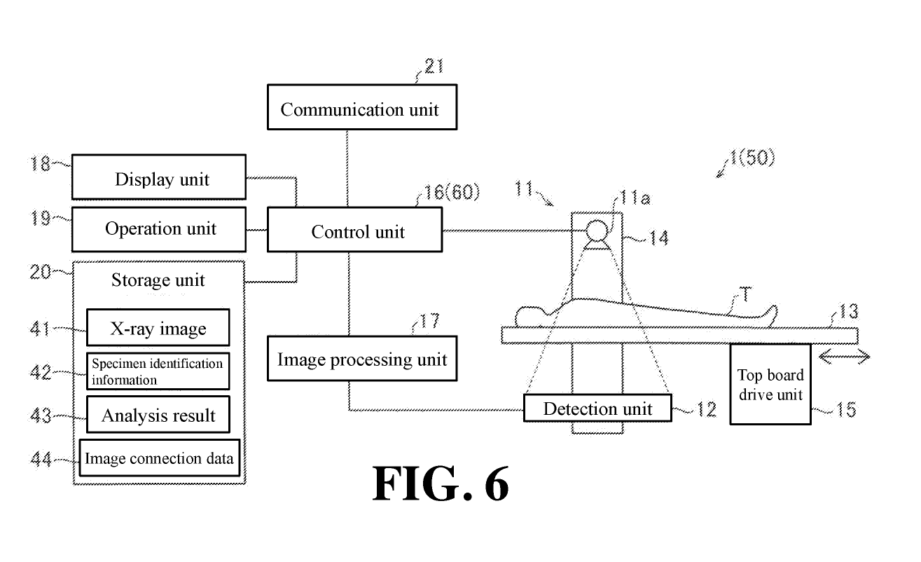

As shown in FIG. 6, the X-ray imaging apparatus 1 is an apparatus for capturing an X-ray imaging for imaging an inside of the subject T by irradiating radiations from the outside of the subject T.

The X-ray imaging apparatus 1 is provided with an irradiation unit 11 for irradiating radiation (X-rays) to the subject T and a detection unit 12 for detecting the radiation transmitted through the subject T. The irradiation unit 11 and the detection unit 12 are arranged so as to face each other across the top board 13 on which a subject T is placed. The irradiation unit 11 and the detection unit 12 are movably supported by the moving mechanism 14. The top board 13 is movable in the horizontal direction by the top board drive unit 15. The irradiation unit 11, the detection unit 12, and the top board 13 are moved via the moving mechanism 14 and the top board drive unit 15 so that the area-of-interest of the subject T can be imaged. The area-of-interest is an area including the collection position P of the specimen sample in the subject T. The X-ray imaging apparatus 1 is provided with a control unit 16 for controlling the moving mechanism 14 and the top board drive unit 15.

The irradiation unit 11 includes a radiation source 11a. The radiation source 11a is, for example, an X-ray tube that generates X-rays by applying a predetermined high voltage. The irradiation unit 11 is connected to the control unit 16. The control unit 16 controls the irradiation unit 11 according to the preset imaging conditions and generates X-rays from the radiation source 11a.

The detection unit 12 detects the X-rays irradiated from the irradiation unit 11 and transmitted through the subject T, and outputs a detection signal corresponding to the detected X-ray intensity. The detection unit 12 is configured by, for example, an FPD (Flat Panel Detector). Further, the X-ray imaging apparatus 1 is provided with an image processing unit 17 that acquires an X-ray detection signal from the detection unit 12 and generates an X-ray image 41 based on the detection signal of the detection unit 12. The detection unit 12 outputs a detection signal of a predetermined resolution to the image processing unit 17.

The image processing unit 17 is a computer composed of, for example, a processor such as a CPU (Central Processing Unit) and storage units such as a ROM (Read Only Memory) and a RAM (Random Access Memory), and functions as an image processing unit by having the processor execute an image processing program. In addition to the generation of the X-ray image 41, the image processing unit 17 can perform correction processing for improving the visibility of the X-ray image 41, synthesis processing for synthesizing a plurality of X-ray images 41, and the like.

The control unit 16 is a computer configured by including a CPU, a ROM, a RAM, and the like. The control unit 16 functions as a control unit that controls each part of the X-ray imaging apparatus 1 by executing a predetermined control program by the CPU. The control unit 16 controls the irradiation unit 11 and the image processing unit 17, and controls the driving of the moving mechanism 14 and the top board drive unit 15. In the second embodiment, the control unit 16 functions as an association means that associates the diagnostic image 40 (X-ray image 41) capable of identifying the collection position P with the specimen identification information 42.

The X-ray imaging apparatus 1 is provided with a display unit 18, an operation unit 19, and a storage unit 20. Further, the X-ray imaging apparatus 1 is provided with a communication unit 21 for connecting with the network N. The display unit 18 is, for example, a monitor such as, e.g., a liquid crystal display. The operation unit 19 is configured to include, for example, a keyboard, a mouse, a touch panel, another controller, or the like. The storage unit 20 is configured by a storage device, such as, e.g., a hard disk drive. The control unit 16 is configured to perform control to display the image generated by the image processing unit 17 on the display unit 18. Further, the control unit 16 is configured to accept an input operation via the operation unit 19. The storage unit 20 is configured to record the data of the X-ray image 41, the data of the specimen identification information 42, the data of the analysis result 43 of the specimen sample, the image connection data 44 to be described later, and the like. The communication unit 21 is configured to be communicable with the specimen analyzing device 2 via the network 6. The communication unit 21 is configured to be connected to the specimen analyzing device 2 one by one without the network 6.

(Specimen Analyzing Device)

The specimen analyzing device 2 is a device that acquires the specimen sample 90 collected from the subject T, measures components necessary for diagnosis, and detects cells. The specimen analyzing device 2 is, for example, a blood analysis device for analyzing blood components, a blood cell classification device, and a chemical analysis device. The object to be measured or detected by the specimen analyzing device 2 varies depending on the type of disease to be diagnosed, so it is selected according to the type of the disease. In the diagnosis of primary aldosteronism, the adrenal vein blood cortisol concentration and/or the aldosterone concentration are measured.

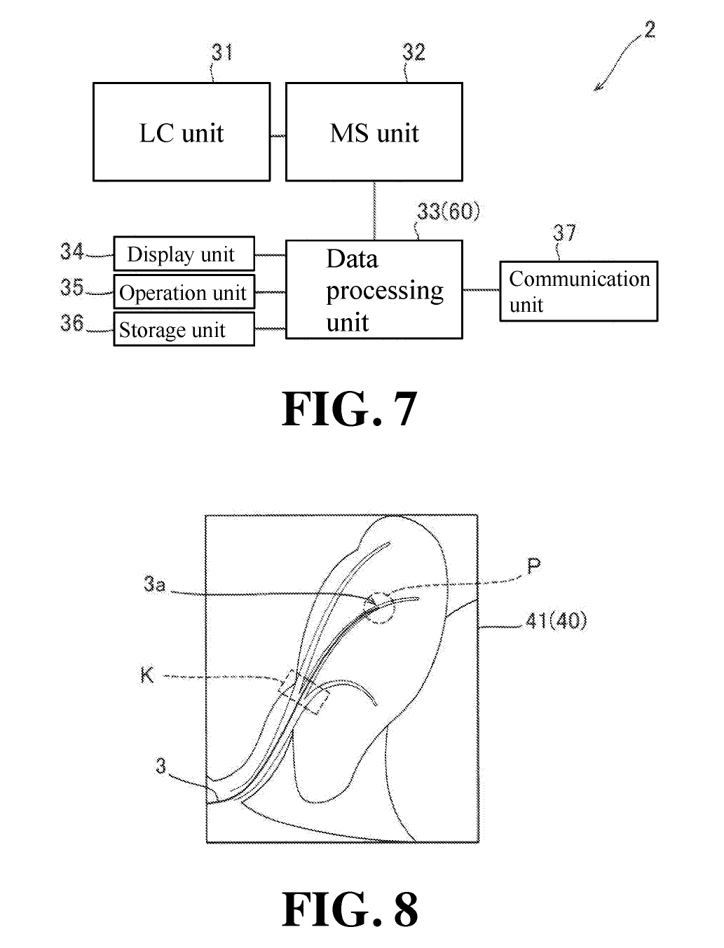

In FIG. 7, as an example of the specimen analyzing device 2, a specimen analyzing device 2 composed of a liquid chromatography mass spectrometer is shown. The specimen analyzing device 2 is provided with a liquid chromatograph unit (hereinafter referred to as “LC unit 31”) for separating a target component contained in the specimen sample 90 and a mass spectrometric unit (hereinafter referred to as “MS unit 32”) that ionizes the separated target component and separates and detects target ions according to the mass number.

The LC unit 31 mainly includes a carrier liquid reservoir which accommodates a carrier liquid, a liquid delivery pump which feeds a carrier liquid together with a specimen sample, a sample introduction unit that introduces the specimen sample, a separation column that separates specimen samples in the carrier liquid for each component.

The MS unit 32 is provided at the subsequent stage of the LC unit 31, and mainly includes an ionization unit for ionizing the sample components separated by the LC unit 31, a mass separator for mass separating the generated ions and passing through specific ions, and an ion detector for detecting ions which passed through the mass separator. The MS unit 32 outputs a detection signal for each mass with respect to the sample components eluted sequentially from the LC unit 31.

The specimen analyzing device 2 is provided with a data processing unit 33 that performs component analysis based on the detection signal of the MS unit 32. The data processing unit 33 conducts a quantitative analysis of a predetermined component (cortisol, aldosterone, etc.) in a specimen sample by preparing a mass spectrum from the detection signal for each mass and comparing it with a known calibration curve.

The specimen analyzing device 2 is provided with a display unit 34, an operation unit 35, a storage unit 36, and a communication unit 37. The display unit 34, the operation unit 35, the storage unit 36, and the communication unit 37 are the same in structure itself as the display unit 18, the operation unit 19, the storage unit 20, and the communication unit 21 of the X-ray imaging apparatus 1, respectively.

(Association of Diagnostic Image and Specimen Identification Information)

In the second embodiment, the control unit 16 acquires the data of the specimen identification information 42, the data of the analysis result 43 of the specimen sample 90, etc., from the specimen analyzing device 2 via the communication unit 21. In other words, the data processing unit 33 of the specimen analyzing device 2 transmits the data of the analysis result 43 and/or the data of the specimen identification information 42 to the X-ray imaging apparatus 1 via the communication unit 37. The control unit 16 associates the received specimen identification information 42 with the X-ray image 41 capable of identifying the collection position P.

In the second embodiment, the association means 60 is configured to further associate the analysis result 43 of the specimen sample 90 with the specimen identification information 42. That is, the control unit 16 is configured to further associate the X-ray image 41 capable of identifying the collection position P with the analysis result 43 of the collected specimen sample 90 via the acquired specimen identification information 42. As described above, the analysis result 43 of the specimen sample includes the component analysis result for the specimen sample and the pathological diagnosis result for the specimen sample.

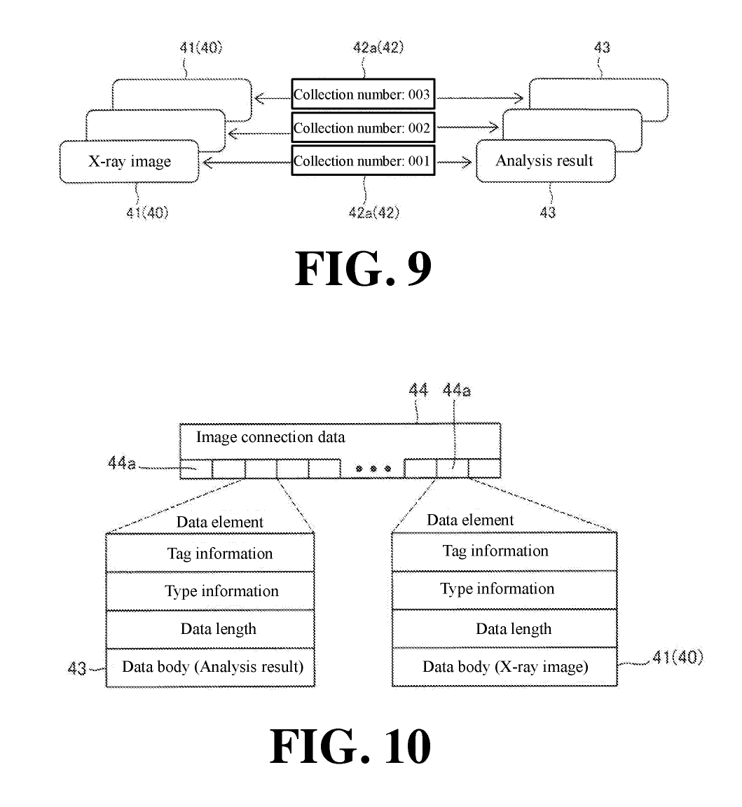

In the second embodiment, an example will be described in which the specimen identification information 42 is a collection number 42a (see FIG. 9) assigned to each collected specimen sample. The collection number 42a is an example of “identification information” recited in claims.

As shown in FIG. 9, the collection number 42a is a unique number assigned each time a sample is collected. In the case of adrenal vein collecting, blood collecting is individually and sequentially performed from a plurality of adrenal veins at different positions. In that case, the collection number 42a is generated as a number, such as, e.g., “001, 002, 003” in the order of sample collection, and is assigned for each specimen sample.

In the second embodiment, the data processing unit 33 of the specimen analyzing device 2 acquires a collection number 42a for each specimen sample 90 to be analyzed when analyzing the specimen sample 90. Then, when the data processing unit 33 generates the analysis result 43 of each specimen sample 90, the data processing unit 33 sends the collection numbers 42a of the specimen samples 90 which has been analyzed and the analysis results 43 to the X-ray imaging apparatus 1 as a set.

With this, the control unit 16 is configured to acquire the specimen identification information 42 (collection number 42a) for each specimen sample 90, together with the analysis result 43 of each specimen sample 90, for a plurality of specimen samples 90 individually collected from a plurality of locations in the subject T during imaging of the X-ray image 41. The control unit 16 associates the X-ray image 41 acquired when each specimen sample 90 is collected with the analysis result 43 of each specimen sample 90 in one-to-one correspondence via the acquired specimen identification information 42 (collection number 42a).

For associating the X-ray image 41 with the analysis result 43, for example, common specimen identification information 42 may be assigned to each of data of the X-ray image 41 and data of the analysis result 43, and the data of the X-ray image 41 and the data of the analysis result 43 may be connected and recorded as single data. In the case of assigning common specimen identification information 42, the X-ray image 41 and the analysis result 43 are managed as individual data linked by the unique specimen identification information 42.

In the second embodiment, the control unit 16 is configured to associate the X-ray image 41 with the analysis result 43 by linking the X-ray image 41 capable of identifying the collection position P of the specimen sample 90 with the analysis result 43 and storing it as a single data file. Specifically, as shown in FIG. 10, the control unit 16 records the X-ray image 41 and the analysis result 43 in the image connection data 44 (DICOM file) in a format conforming to the DICOM standard.

In principle, the image connection data 44 (DICOM file) is composed of a set of data elements 44a including tag information, type information, data length, and a data body. The tag information indicates the type of information stored as the data body. The type information indicates the data format (character string or numeric value) of the data body. The data length indicates the information amount of the data body. The data of the X-ray image 41 and the data of the analysis result 43 are stored as the data body.

The control unit 16 generates the image connection data 44 including the data element 44a that stores the X-ray image 41 and the data element 44a that stores the analysis result 43. As a result, a single data file (image connection data 44) in which the X-ray image 41 and the analysis result 43 are connected is recorded. When a doctor or the like browses the image connection data 44, the collection position P of the specimen sample 90 indicated by the X-ray image 41 and the analysis result 43 of the specimen sample 90 can be browsed together.

(Associating Processing)

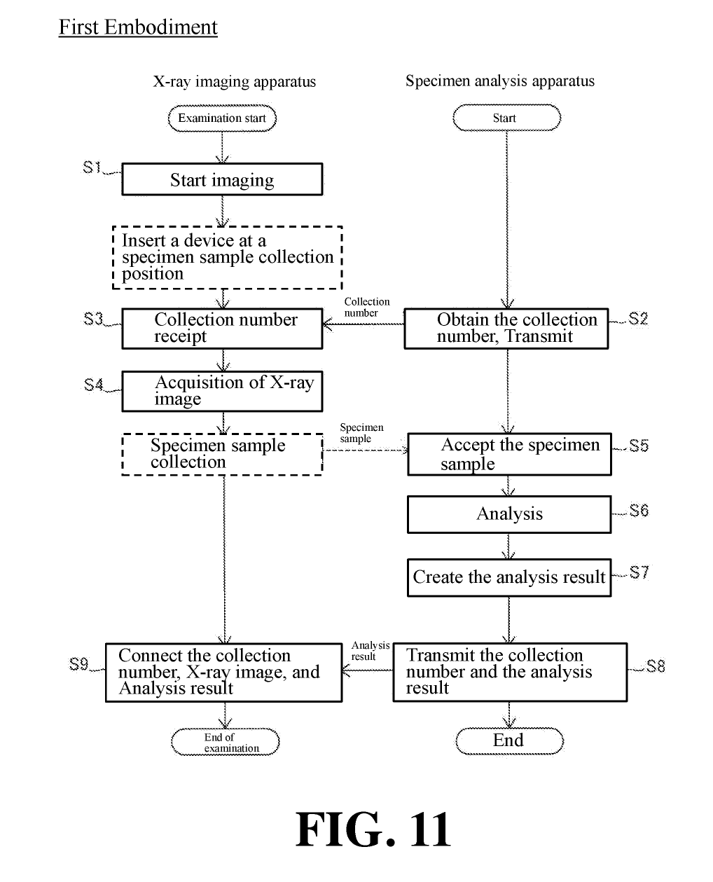

Next, with reference to FIG. 11, the flow of associating processing between the X-ray image 41 and the analysis result 43 by the diagnostic imaging system 100 (X-ray imaging apparatus 1 and specimen analyzing device 2) will be described.

Upon starting the examination, in Step S1, the X-ray imaging apparatus 1 starts imaging the X-ray image and displays the fluoroscopic image of the subject T in the moving image format on the display unit 18.

Using the image displayed on the display unit 18, a doctor inserts the specimen collection device 3 into the subject T and sends it to the collection position P of specimen sample 90. That is, the tip end portion 3a of the specimen collection device 3 (catheter) is put in one of adrenal veins. The specimen collection device 3 is indwelled at the collection position P until collection of the specimen sample 90 is completed.

In Step S2, the specimen analyzing device 2 acquires the collection number 42a of the specimen sample 90, and transmits the acquired collection number 42a from the data processing unit 33 to the control unit 16. The collection number 42a may be obtained, for example, by accepting an input operation via the operation unit 35. Further, the data processing unit 33 may automatically generate the collection number 42a for each order of accepting the specimen sample 90 is to be analyzed after starting to collect the specimen sample 90 (after having the specimen analyzing device 2 standby).

In Step S3, the control unit 16 of the X-ray imaging apparatus 1 accepts the collection number 42a transmitted from the specimen analyzing device 2. In Step S4, the control unit 16 of the X-ray imaging apparatus 1 acquires the X-ray image 41 when the specimen sample 90 is collected. That is, the control unit 16 records the X-ray image 41 as a still image in the storage unit 20 at a predetermined timing out of X-ray images in a moving image format. As shown in FIG. 8, the X-ray image 41 shows the specimen collection device 3 at the collection position P of the specimen sample 90, and the collection position P of the specimen sample is acquired as an identifiable image. Further, the control unit 16 assigns a collection number 42a to the X-ray image 41. That is, the control unit 16 associates the X-ray image 41 with the collection number 42a by recording the X-ray image 41 when the specimen sample 90 is collected in association with the collection number 42a.

Here, the operator of the specimen collection device 3 operates the specimen collection device 3 to collect a specimen sample 90. That is, the operator performs first collection of adrenal vein blood by the catheter indwelled at the collection position P.

In Step S5, the specimen analyzing device 2 accepts the collected specimen sample 90. That is, the specimen sample 90 acquired by the specimen collection device 3 is supplied to the specimen analyzing device 2 directly or via a specimen container 4. The accepted specimen sample 90 is identified by the collection number 42a.

In Step S6, the specimen analyzing device 2 analyzes the accepted specimen sample 90. That is, the data processing unit 33 performs a quantitative analysis of a predetermined component (cortisol, aldosterone, etc. in the case of diagnosis of primary aldosteronism) in the specimen sample based on the detection signal. In Step S7, the data processing unit 33 creates an analysis result 43. The data processing unit 33 creates the data of predetermined items, such as, e.g., a cortisol concentration and an aldosterone concentration in specimen sample, as analysis result 43. The data processing unit 33 associates the analysis result 43 of the specimen sample 90 with the collection number 42a by recording the analysis result 43 of the specimen sample 90 in association with the collection number 42a.

When the analysis result 43 is obtained, in Step S8, the data processing unit 33 transmits the analysis result 43 and the collection number 42a of the specimen sample 90 to the X-ray imaging apparatus 1.

Upon receiving the data transmission, the X-ray imaging apparatus 1 associates the analysis result 43 with the X-ray image 41 based on the acquired collection number 42a in Step S9. That is, the control unit 16 connects the analysis result 43 and the X-ray image 41 which match the collection number 42a to generate a single image connection data 44.

Although omitted in FIG. 11, in the case of the adrenal vein collecting for the diagnosis of primary aldosteronism, after the first specimen sample 90 is collected, the operator of the specimen collection device 3 again places the specimen collection device 3 at the next blood collection position (another adrenal vein) with the fluoroscopic image (moving image) as a clue, and performs blood collecting. Therefore, each time the specimen collection device 3 is placed at the blood collection position, the processing in Steps S2 to S9 is repeated.

As a result, even when specimen samples 90 are sequentially collected from a plurality of collection positions P, the control unit 16 generates image connection data 44 by associating the collection numbers 42a with the X-ray images 41 indicating the respective collection positions P and the corresponding analysis results 43. The image connection data 44 is generated by the number of collected specimen samples 90.

### Effects of Second Embodiment

In the second embodiment, the following effects can be obtained.

In the second embodiment, in the same manner as in the first embodiment, by associating the diagnostic image 40 (X-ray image 41) capable of identifying the collection position P with the specimen identification information 42, it becomes possible to reduce the management burden of the analysis result and the collection position P of the specimen sample 90 when performing the diagnosis by the specimen sample 90 collected from the subject T.

Further, in the second embodiment, as described above, the specimen identification information 42 collected from the specimen contains the collection number 42a assigned for each specimen sample 90 at the time of collection. With this, by assigning the unique collection number 42a for each specimen sample when the specimen sample 90 is collected, it becomes possible to easily and assuredly associate the collection position P of the specimen sample 90 with the X-ray image 41 capable of identifying the collection position P of the specimen sample 90.

Further, in the second embodiment, as described above, the specimen identification information 42 collected from the subject includes the collection number 42a received from the specimen analyzing device 2 which analyzes the specimen sample 90. With this, the collection number 42a can be easily obtained from the specimen analyzing device 2 and the automatic association can be performed, which can improve the convenience of the diagnostic imaging system 100.

Further, in the second embodiment, as described above, the association means 60 is configured to associate the analysis result 43 of the specimen sample 90 with the specimen identification information 42 collected from the subject. This makes it possible to manage the X-ray image 41 capable of identifying the collection position P and the analysis result 43 of the specimen sample 90 collected from the collection position P in a one-to-one correspondence. Therefore, the management burden of the analysis result 43 and the collection position P of the specimen sample 90 can be further reduced.

In the second embodiment, as described above, the analysis result 43 of the specimen sample 90 includes the pathological diagnosis result for the specimen sample 90. With this, when the existence and absence of a lesion is determined and/or the type of a lesion is identified by the pathological diagnosis result, it becomes possible to directly grasp the lesion part (collection position P of the specimen sample 90) from the X-ray image 41. As a result, it becomes possible to facilitate the grasp of the lesion part and to improve the convenience of the diagnostic imaging system 100.

In the second embodiment, as described above, the analysis result 43 of the specimen sample 90 includes the component analysis result for the specimen sample 90. With this, for example, even in cases where a lesion or the like is collected from a plurality of locations around the examination target part, it is possible to manage the component analysis result and the collection position P of each specimen sample 90 in association with each other. As a result, the management burden of the analysis result 43 and the collection position P can be effectively reduced.

### Third Embodiment

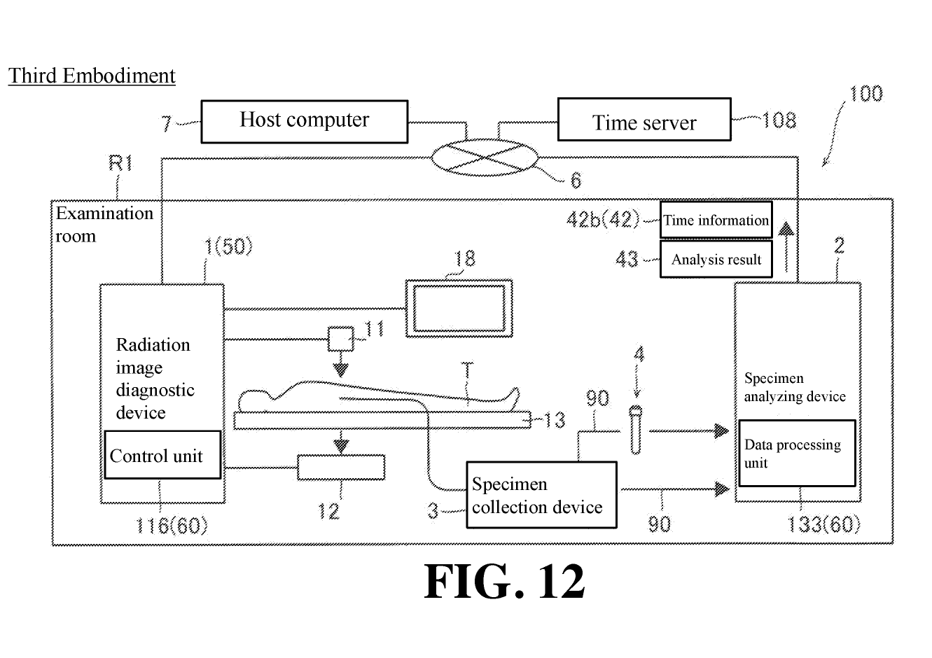

Next, a third embodiment will be described with reference to FIG. 12 to FIG. 14. In this third embodiment, unlike the above-described second embodiment using the collection number 42a as the specimen identification information 42, an example of using the time information 42b as the specimen identification information 42 will be described. In the third embodiment, the same reference numerals are allotted to the common configurations as those of the first embodiment and the second embodiment, and the description thereof will be omitted.

(Association of X-Ray Image and Analysis Result)

As shown in FIG. 12, in the third embodiment, the X-ray imaging apparatus 1 and the specimen analyzing device 2 are connected to the time server 108 via the network 6. That is, the control unit 116 of the X-ray imaging apparatus 1 and the data processing unit 133 of the specimen analyzing device 2 can be operated synchronously in time by the common time server 108. The control unit 116 and the data processing unit 133 are examples of the “association means” recited in in claims.

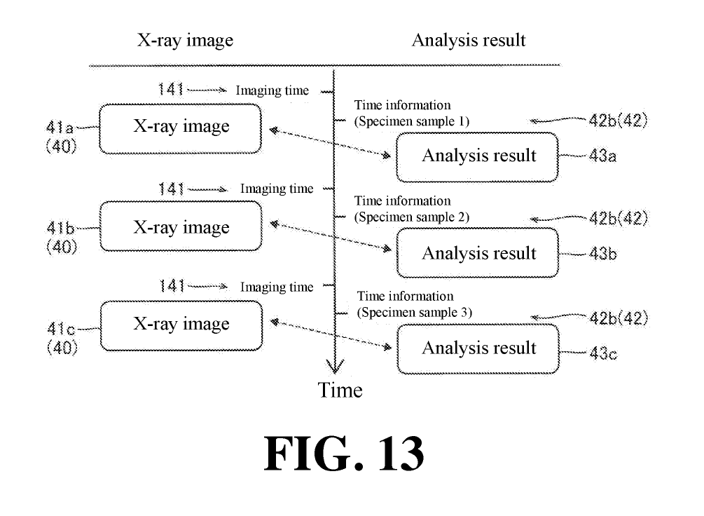

In the third embodiment as shown in FIG. 13, the control unit 116 is configured to acquire the time information 42b together with the analysis result 43 of the specimen sample and to associate the corresponding X-ray image 41 with the analysis result 43 on the basis of the acquired time information 42b and the imaging time of the X-ray image 41. Note that the time information 42b is an example of the “identification information” recited in claims.

Specifically, as shown in FIG. 13, when acquiring the X-ray image 41 (still image) when the specimen sample 90 is collected, the control unit 116 is configured to record the imaging time information 141 (photographing time) that acquired the X-ray image 41 in data of the X-ray image 41. Therefore, the individual X-ray image 41 acquired by the X-ray imaging apparatus 1 can be uniquely identified based on the imaging time information 141 included in the image data.

When the specimen sample 90 is accepted and the sample analysis is started, the data processing unit 133 (see FIG. 12) of the specimen analyzing device 2 is configured to acquire the time when the analysis is started as the time information 42b and record it so as to be included in the analysis result 43 of the specimen sample. For this reason, the individual analysis results 43 created by the specimen analyzing device 2 can identify which specimen sample analysis result is based on the time information 42b.

Therefore, in the diagnostic imaging system 100 shown in FIG. 12, when the specimen sample 90 is collected from a plurality of adrenal veins in order, the order of collecting the specimen sample 90, the order of acquiring the X-ray image 41, and the order of starting the sample analysis are coincided with each other. When collecting specimen samples 90 from a plurality of collection positions in a subject T, it involves a moving operation of the specimen collection device 3 such as a catheter. Therefore, it is difficult to collect it continuously in time. For this reason, between collections of the respective specimen samples 90, there is a sufficient time interval to accurately identify the correspondence relation of the above-described sample collection order, image acquisition order, and analysis start order.

Therefore, the control unit 116 is configured to identify the X-ray image 41 indicating the collection position P of the specimen sample 90 and the analysis result 43 of the specimen sample 90 collected at the collection position P and associates them with each other by collating the time information 42b acquired together with the analysis result 43 and the time series of imaging times of a series of X-ray images 41.

For example, as shown in FIG. 13, in cases where the acquired time information 42b is before the imaging time of the X-ray image 41a when the specimen sample 90 is collected and before the imaging time of the X-ray image 41b when the specimen sample 90 is next collected, the control unit 116 is configured to mutually associate the X-ray image 41a, the time information 42b, and the analysis result 43. In FIG. 13, since the time information 42b of the analysis result 43a is between the imaging time of the X-ray image 41a and the imaging time of the X-ray image 41b, the analysis result 43a (time information 42b) is associated with the X-ray image 41a. Likewise, the X-ray image 41b and the analysis result 43b are associated with each other, and the X-ray image 41c and the analysis result 43c are associated with each other.

(Associating Processing)

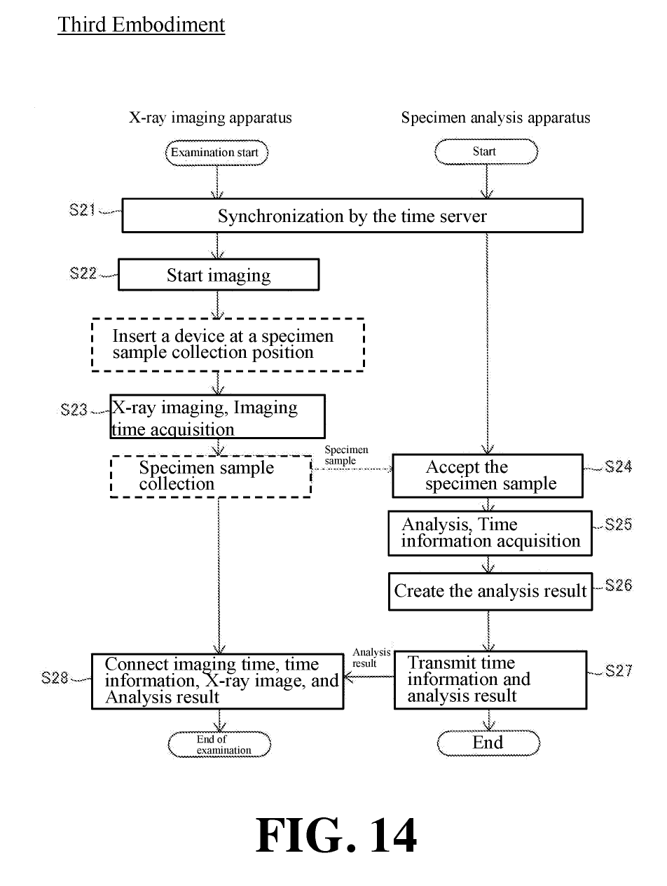

As shown in FIG. 14, in the third embodiment, first, in Step S21, the X-ray imaging apparatus 1 (control unit 116) and the specimen analyzing device 2 (data processing unit 133) can be operated synchronously in time by the time server 108. That is, a time adjustment is performed.

In Step S22, the X-ray imaging apparatus 1 starts image capturing, and the fluoroscopic image of the subject T is displayed in moving unit format on the display unit 18. When the specimen collection device 3 is placed at the collection position P, in Step S23, the specimen analyzing device 2 acquires the X-ray image 41 when the specimen sample is collected. At this time, the X-ray image 41 is recorded so as to include the imaging time information 141 (imaging time).

When the specimen sample 90 is collected by the specimen collection device 3, in Step S24, the specimen analyzing device 2 accepts the collected specimen sample 90. In Step S25, the specimen analyzing device 2 analyzes the accepted specimen sample 90. At this time, the data processing unit 133 acquires the time information 42b indicating the start time of the sample analysis. In Step S26, the data processing unit 133 creates an analysis result 43. The data processing unit 133 associates the analysis result 43 of the specimen sample 90 with the time information 42b identifying the specimen sample 90 by recording the analysis result 43 of the specimen sample 90 so as to include the time information 42b.

When the analysis result 43 is obtained, in Step S27, the data processing unit 133 transmits the analysis result 43 and the time information 42b of the specimen sample 90 to the X-ray imaging apparatus 1. Note that since it takes time to complete the analysis, the transmission of the analysis result 43 and the acquisition of the next X-ray image 41 (the processing of Step S23 on the second specimen sample) may sometimes vary in anteroposterior relation. Even in such a case, as shown in FIG. 13, the corresponding X-ray image 41 can be identified based on the anteroposterior relation between the imaging time and the analysis start time (time information 42b).

In Step S28, the X-ray imaging apparatus 1 which received the data transmission associates the analysis result 43 to which the time information 42b is assigned with the X-ray image 41 based on the acquired time information 42b and the imaging time (imaging time information 141) of the X-ray image 41. The control unit 16 connects the analysis result 43 and the X-ray image 41 identified based on the time series relation between the time information 42b and the imaging time to generate single image connection data 44.

Note that each time the specimen collection device 3 is placed at the second and subsequent blood collection positions, the processing in Steps S23 to S28 is repeated. The control unit 16 associates the X-ray image 41 indicating each collection position P with the corresponding analysis result 43 (time information 42b) and generates it as image connection data 44.

### Effects of Third Embodiment

In the third embodiment, in the same manner as in the first embodiment, by associating the diagnostic image 40 (X-ray image 41) capable of identifying the collection position P with the specimen identification information 42, it becomes possible to reduce the management burden of the analysis result and the collection position P of the specimen sample 90 when performing the diagnosis by the specimen sample 90 collected from the subject T.

Further, in the third embodiment, as described above, as the specimen identification information 42, the time information 42b which analyzed the specimen sample 90 is used. This makes it possible to easily perform the process of automatically associating the X-ray image 41 with the analysis result 43 by the time information 42b acquired by the specimen analyzing device 2.

### Fourth Embodiment

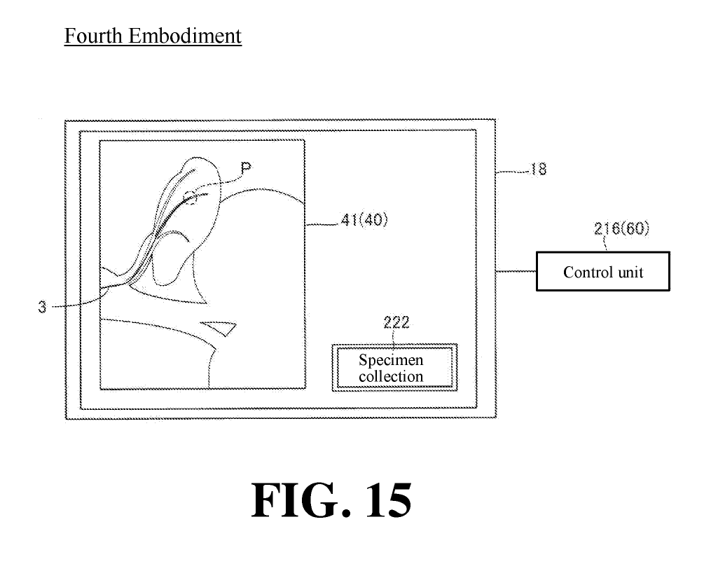

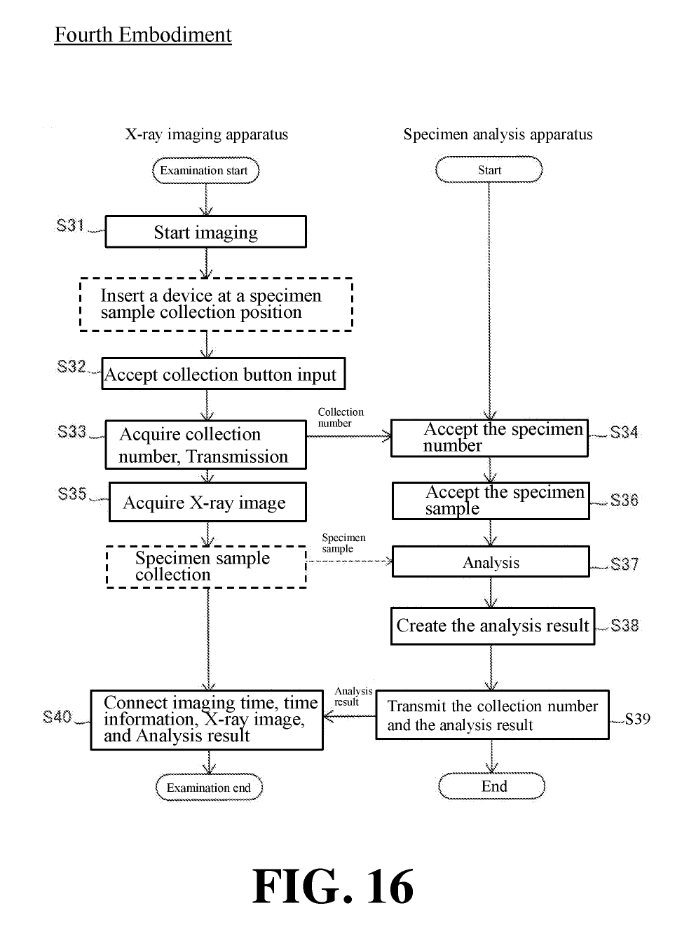

Next, a fourth embodiment will be described with reference to FIG. 15 to FIG. 16.

In this fourth embodiment, unlike the above-described second embodiment in which the specimen analyzing device 2 acquires the collection number 42a and transmits it to the X-ray imaging apparatus 1, an example in which the X-ray imaging apparatus 1 acquires the collection number 42a will be described. In the fourth embodiment, the same reference numerals are allotted to the common configurations as those of the first embodiment and the second embodiment, and the description thereof will be omitted.

(Association of X-Ray Image and Analysis Result)

In the fourth embodiment, the collection number 42a similar to that of the second embodiment is used for the specimen identification information 42. The control unit 216 (see FIG. 15) assigns the collection number 42a to the X-ray image 41 capable of identifying the collection position P of the specimen sample 90 when the specimen sample 90 is collected, acquires the collection number 42a together with the analysis result 43 of the specimen sample 90, and associates the analysis result 43 with the X-ray image 41 based on the acquired collection number 42a (see FIG. 9). Note that the control unit 216 is an example of the “association means” recited in claims.

Here, in the fourth embodiment, as shown in FIG. 15, the control unit 216 is configured to assign the collection number 42a to the X-ray image 41 based on the operation input accepted via the operation unit 19 when the specimen sample 90 is collected.

For example, the control unit 216 sets a specimen collection button 222 (icon) on the display screen of the display unit 18 shown in FIG. 15. A specimen collection button (not shown) as a physical input device may be provided in the operation unit 19.

In the fourth embodiment, when the specimen collection device 3 is placed at the collection position P and the collection of the specimen sample 90 is started, the operator performs an input operation by the specimen collection button 222. Based on the operation input, the control unit 216 generates a collection number 42a and transmits it to the specimen analyzing device 2. With this, the control unit 216 associates the X-ray image 41 with the analysis result 43 based on the collection number 42a transmitted together with the analysis result 43 from the specimen analyzing device 2.

(Associating Processing)

As shown in FIG. 16, in the fourth embodiment, in Step S31, the X-ray imaging apparatus 1 starts X-ray imaging, and the fluoroscopic image of the subject T is displayed in a moving image format on the display unit 18. When the specimen collection device 3 is placed at the collection position P, in Step S32, the control unit 216 receives an operation input via the operation unit 19. That is, the control unit 216 accepts the input operation of the specimen collection button 222 by the operator.

Upon accepting the input operation of the specimen collection button 222, the control unit 216 acquires (generates) the collection number 42a of the present specimen sample 90 and transmits it to the specimen analyzing device 2 in Step S33. In Step S34, the specimen analyzing device 2 accepts the collection number 42a.

In Step S35, the control unit 216 acquires the X-ray image 41 when the specimen sample is collected. At this time, the control unit 216 assigns the collection number 42a acquired in Step S33 to the X-ray image 41.

The processing of Steps S36 to S40 is similar to Steps S5 to S9 in the associating processing of the second embodiment, and therefore the description thereof will be omitted.

### Effects of Fourth Embodiment

The effects of the fourth embodiment are the same as those of the second embodiment.

### First Embodiment

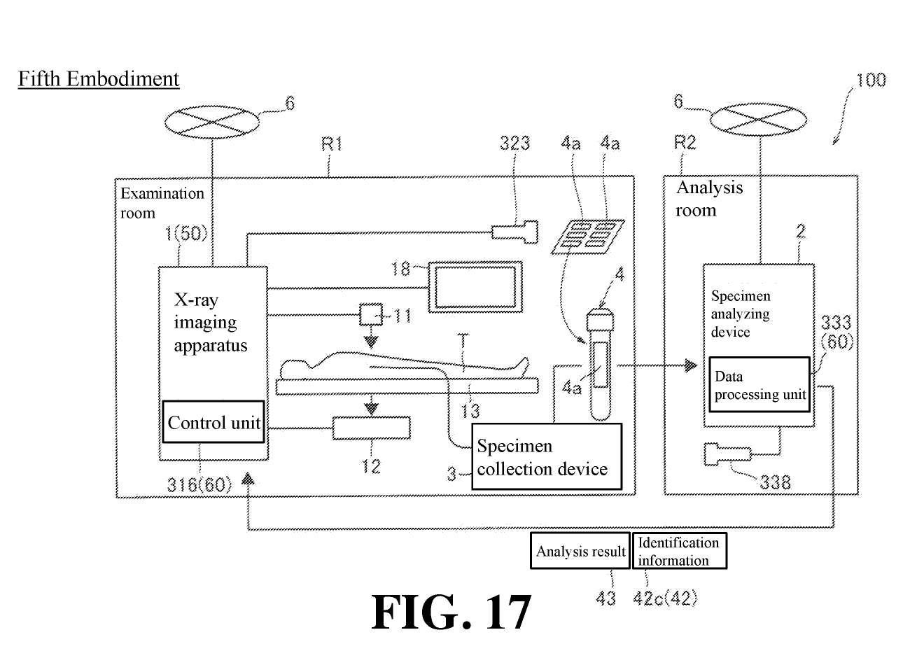

Next, a firth embodiment will be described with reference to FIG. 17 to FIG. 19. In this fifth example, unlike the above-described second embodiment using the collection number 42a as the specimen identification information 42 and the above-described third embodiment using the time information 42b, an example using identification information 42c to be attached to the specimen container 4 for accommodating the specimen sample 90 collected as the specimen identification information 42 will be described. In the fifth embodiment, the same reference numerals are allotted to the common configurations as those of the first embodiment, and the description thereof will be omitted.

(Association of X-Ray Image and Analysis Result)

In the fifth embodiment, the X-ray imaging apparatus 1 and the specimen analyzing device 2 do not have to be configured to be able to transmit and receive the specimen identification information 42 by the network 6 such as a LAN. For example, as shown in FIG. 17, it may be configured such that the X-ray imaging apparatus 1 and the specimen analyzing device 2 are separately installed in the examination room R1 and the analysis room R2, respectively, and are not allowed to transmit and receive the specimen identification information 42. Further, even in cases where the X-ray imaging apparatus 1 and the specimen analyzing device 2 are connected to the network 6, it may be configured such that the host computer 7 is allowed to transmit and receive date (see FIG. 1) and the date exchange is not allowed between the X-ray imaging apparatus 1 and the specimen analyzing device 2.

In the fifth embodiment, the specimen identification information 42 is identification information 42c to be attached to the specimen container 4 for accommodating a collected specimen. The identification information 42c is, for example, a specimen ID to be attached to a specimen container 4 in the form of a barcode or a two-dimensional code. The identification information 42c is prepared, for example, in the form of a label 4a printed with a barcode, and is attached to the specimen container 4 by an operator when a specimen sample 90 is collected. With this, the identification information 42c is used to identify the specimen sample 90.

In the fifth embodiment, the X-ray imaging apparatus 1 is provided with a reading unit 323 for reading the identification information 42c attached to the specimen container 4 for accommodating a collected specimen sample 90. Further, the specimen analyzing device 2 is also provided with a reading unit 338. The reading units 323 and 338 each are, for example, a bar code reader (two-dimensional code reader) corresponding to the identification information 42c, and are capable of reading the identification information 42c attached to the specimen container 4.

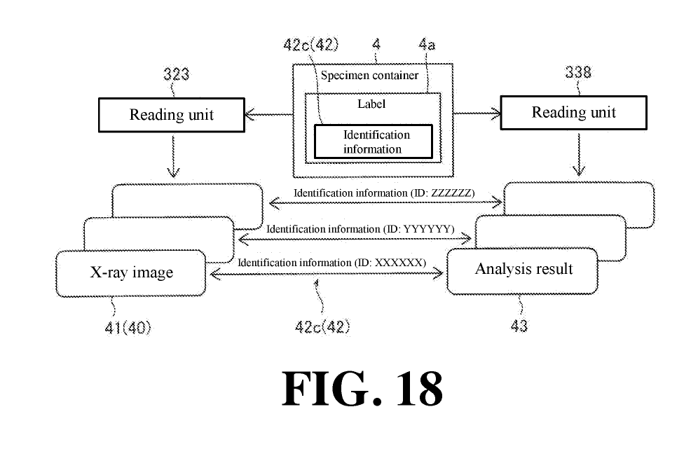

In the fifth embodiment, the control unit 316 is configured to give the identification information 42c read by the reading unit 323 to the X-ray image 41 when the specimen sample 90 is collected. Then, the control unit 316 acquires the analysis result 43 to which the identification information 42c is attached. With this, as shown in FIG. 18, the control unit 316 is configured to associate the X-ray image 41 with the analysis result 43 based on the identification information 42c assigned to each of each of the X-ray image 41 and the analysis result 43. Note that the control unit 316 is an example of the “association means” recited in claims.

Further, as shown in FIG. 17, the specimen analyzing device 2 (data processing unit 333) is configured to assign the identification information 42c read out by the reading unit 338 to the analysis result 43 when performing the sample analysis. With this, the analysis result 43 and the X-ray image 41 are associated with each other via common identification information 42c. Note that the data processing unit 333 is an example of the “association means” recited in claims.

(Associating Processing)

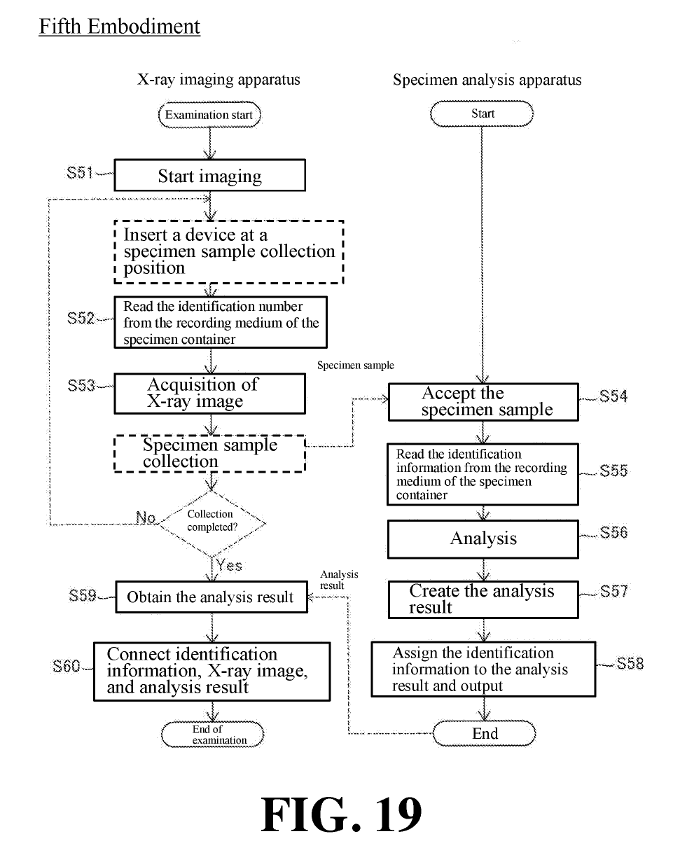

As shown in FIG. 19, in the fifth embodiment, in Step S51, the X-ray imaging apparatus 1 starts X-ray imaging, and the fluoroscopic image of the subject T is displayed in a moving image format on the display unit 18. When the specimen collection device 3 is placed at a collection position P, in Step S52, the identification information 42c is read by the reading unit 323, and the control unit 316 acquires the identification information 42c. That is, an operator selects an arbitrary label 4a (see FIG. 17) on which the identification information 42c has been printed using the reading unit 323 and reads the identification information 42c. The label 4a from which the identification information 42c has been read is affixed to the specimen container 4 for accommodating the present specimen sample 90 by the operator.

In Step S53, the control unit 316 acquires an X-ray image 41 (still image) when the specimen sample 90 is collected. At this time, the control unit 316 assigns the identification information 42c acquired in Step S52 to the X-ray image 41 and records it.

The specimen sample is accommodated in the specimen container 4. The specimen container 4 accommodating the specimen sample 90 is transported by the operator to the analysis room R2 where the specimen analyzing device 2 is installed.

Steps S52 to S53 are repeated until all the specimen samples 90 required for this adrenal vein collecting are collected.

On the other hand, the specimen analyzing device 2 accepts the specimen sample 90 in Step S54. That is, the specimen container 4 accommodating the specimen sample 90 is set in the specimen analyzing device 2. In Step S55, the identification information 42c is read by the reading unit 338, and the data processing unit 333 acquires the identification information 42c. That is, the operator reads the identification information 42c attached to the specimen container 4 using the reading unit 338.

In Step S56, the specimen analyzing device 2 analyzes the accepted specimen sample 90. In Step S57, the data processing unit 333 creates an analysis result 43. In Step S58, the data processing unit 333 assigns the identification information 42c to the analysis result 43 of the specimen sample 90 and outputs it.

Then, in Step S59, the control unit 316 of the X-ray imaging apparatus 1 acquires the analysis result 43 to which the identification information 42c is assigned. The passing method of the data of the analysis result 43 including the identification information 42c is arbitrary. For example, when transmission and reception of data with respect to the host computer 7 (see FIG. 1) is permitted for each of the X-ray imaging apparatus 1 and the specimen analyzing device 2, the data of the analysis result 43 output by the specimen analyzing device 2 to the host computer 7 may be acquired by the X-ray imaging apparatus 1 from the host computer 7. For example, the specimen analyzing device 2 may output the data of the analysis result 43 to a portable recording medium, such as, e.g., an optical disc or a flash memory, and the X-ray imaging apparatus 1 may read the data from the portable recording medium.

In Step S60, the control unit 316 of the X-ray imaging apparatus 1 associates the analysis result 43 with the X-ray image 41 based on the acquired identification information 42c. That is, the control unit 316 connects the analysis result 43 and the X-ray image 41 in which the identification information 42c matches.

### Effects of First Embodiment

In the fifth embodiment, in the same manner as in the first embodiment, by associating the diagnostic image 40 (X-ray image 41) capable of identifying the collection position P with the sample identification information 42, it becomes possible to reduce the management burden of the analysis result 43 and the collection position P of the specimen sample 90 when performing the diagnosis by the specimen sample 90 collected from the subject T.

Further, in the fifth embodiment, as described above, the specimen identification information 42 is identification information 42c to be attached to the specimen container 4 for accommodating a collected specimen sample 90. With this, when the specimen sample 90 is collected, only by inputting (reading) the identification information 42c attached to the specimen container 4, it is easily to associate the diagnostic image 40 with the identification information 42c.

### Sixth Embodiment

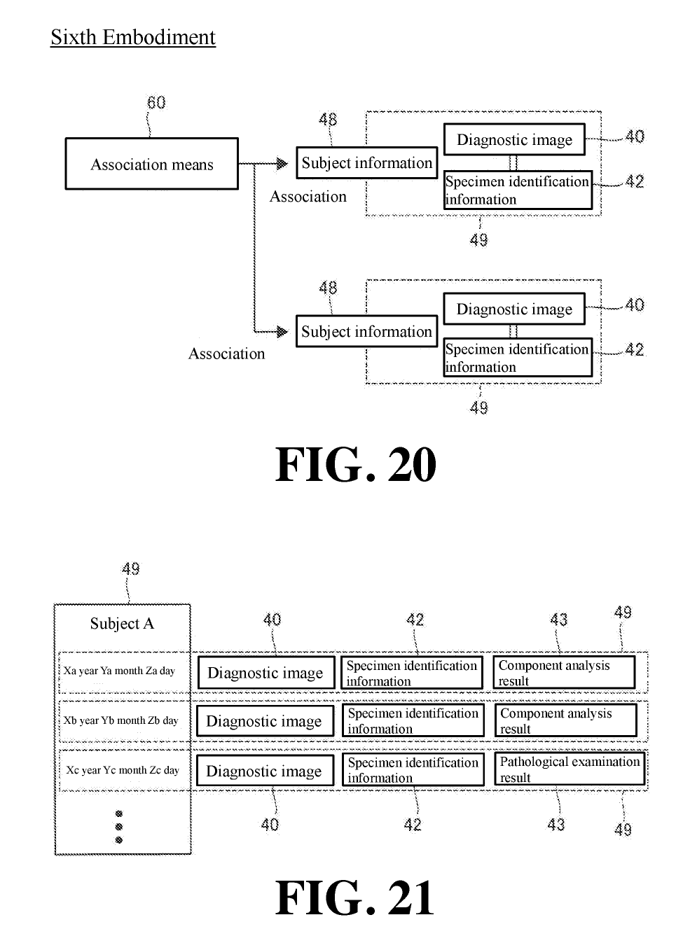

Next, a sixth embodiment will be described with reference to FIG. 20 and FIG. 21.

In this sixth embodiment, an example will be described in which in addition to the association between the specimen identification information 42 and the diagnostic image 40 (X-ray image 41) performed in the first to fifth embodiments, association of the information identifying the subject T is further performed. In the sixth embodiment, the same reference numerals are allotted to the common configurations as those of the first embodiment, and the description thereof will be omitted.

As shown in FIG. 20, in the sixth embodiment, the association means 60 is configured to further associate information identifying the subject T (hereinafter referred to as “subject information 48”) with each of a plurality of diagnostic images 40 associated with the specimen identification information 42.

The subject information 48 is identification information identifying the individual subject T. The subject information 48 may be, for example, a patient ID assigned to each individual subject T, but it is not particularly limited as long as it can identify the subject T. The subject information 48 is recorded, for example, in the host computer 7 of the facility, and is used as identification information for managing past medical records, electronic medical record data, etc., for each patient.

The association means 60 further associates the subject information 48 when associating the specimen identification information 42 with the diagnostic image 40. As a result, when collection of a specimen sample 90 and generation of a diagnostic image 40 capable of identifying the collection position P are performed at different time points, such as periodic examinations, a data group 49 of the specimen identification information 42, the diagnostic image 40, and the subject information 48, which are associated with each other, is generated every time the examination is performed. These data groups 49 may be generated as a single file in the form of image connection data 44 (see FIG. 10) including the subject information 48.

As a result, as shown in FIG. 21, the data group 49 generated each time the examination is performed is mutually associated via the common subject information 48, it becomes possible to manage them collectively. FIG. 21 shows an overview of the data management in which a plurality of data groups 49 (only three shown) associated by the common subject information 48 are arranged in order of time series (year, month, day). Each data group 49 includes the specimen identification information 42 of the specimen sample 90 collected at each examination and the diagnostic image 40 capable of identifying the collection position P, the analysis result 43 of the specimen sample 90, and the like. As a result, when a doctor observes the subject T, the doctor can refer to the examination date and time of each examination, the collection position P of the specimen sample 90 in each examination, and the analysis result 43 of the specimen sample 90 obtained in the examination in a manner summarized for each subject T.

### Effects of Sixth Embodiment

In the sixth embodiment, in the same manner as in the first example, by associating the diagnostic image 40 (X-ray image 41) capable of identifying the collection position P with the specimen identification information 42, it becomes possible to reduce the management burden of the analysis result and the collection position P of the specimen sample 90 when performing the diagnosis by the specimen sample 90 collected from the subject T.

In the sixth embodiment, as described above, the association means 60 is configured to further associate the subject information 48 with each of the plurality of diagnostic images 40 associated with the specimen identification information 42. With this configuration, when the association between the collected specimen sample 90 and the diagnostic image 40 which identifies the collection position P is performed multiple times on the same subject T, each diagnostic image 40 (and specimen sample 90) can be managed collectively by the subject information 48. This makes it possible to easily grasp multiple examination results temporary spaced apart with respect to the same subject T in time series easily, so that the patient (subject T) follow-up observation can be facilitated.

### Seventh Embodiment

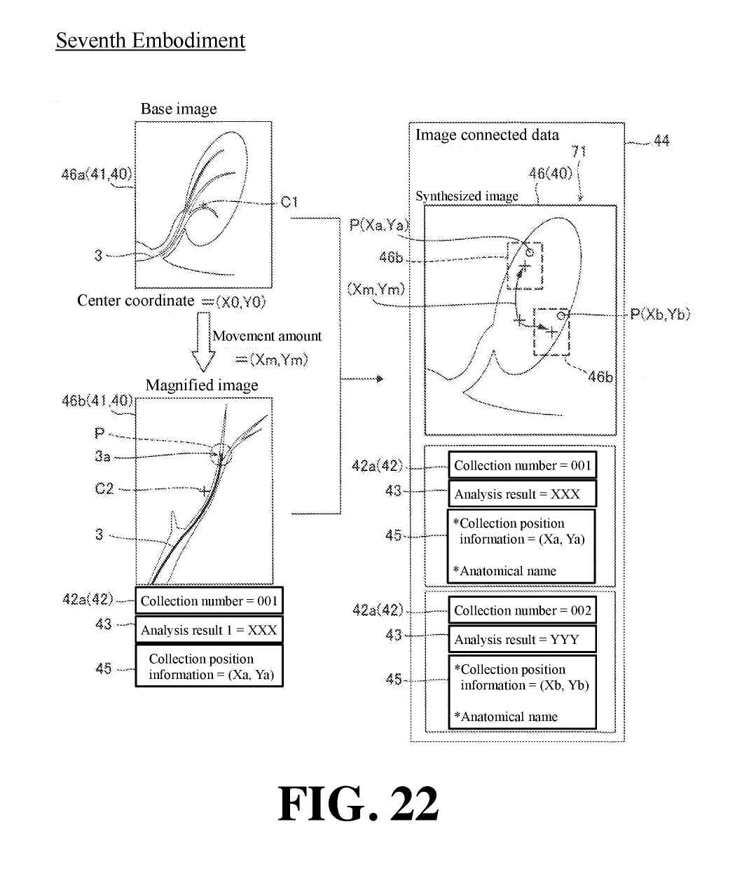

Next, a seventh embodiment will be described with reference to FIG. 5 and FIG. 22.

In this seventh embodiment, unlike the first to sixth embodiments in which the X-ray image 41 is associated with the specimen identification information 42, an example will be described in which, in addition to the X-ray image 41 and the specimen identification information 42, association of the collection position information 45 is performed. In the seventh embodiment, the same reference numerals are allotted to the common configurations as those of the second embodiment (see FIG. 5 to FIG. 7), and the description thereof will be omitted.

(Association of X-Ray Image and Collection Position Information)

In the seventh embodiment, the association between the X-ray image 41 and the specimen identification information 42 and the analysis result 43 may be performed by any of the first to sixth configurations described above. Here, an example of the configuration of the second embodiment using the collection number 42a will be described as an example. In the seventh embodiment, the association means 60 further associate the information (hereinafter referred to as “collection position information 45”) (see FIG. 22) which identifies the collection position P of the specimen sample 90 in the diagnostic image 40 with the diagnostic image 40 when the specimen sample 90 is collected.

Note that the association means 60 may associate the collection position information 45 with the specimen identification information 42. It is sufficient that the collection position information 45 is associated with one of the diagnostic image 40 and the specimen identification information 42. However, in the seventh embodiment, an example is shown in which the collection number 42a is used as the specimen identification information 42, and the collection position information 45 is associated with both the diagnostic image 40 and the specimen identification information 42.

In the example shown in FIG. 22, the control unit 16 is configured to further acquire the collection position information 45 of the specimen sample 90 in the X-ray image 41 when the specimen sample 90 is collected. The control unit 16 is configured to further acquire the collection position information 45 of the specimen sample 90 in the X-ray image 41 when the specimen sample 90 is collected.

The collection position information 45 of the specimen sample 90 in the X-ray image 41 can be obtained by, for example, image processing. In this case, the control unit 16 (see FIG. 5) controls the image processing unit 17 (see FIG. 6) to detect the position where the tip end portion 3a of the specimen collection device 3 is indwelled in the X-ray image 41 by image recognition. For image recognition, any known methods such as template matching, filter processing for detecting a tip end portion, pattern recognition using machine learning, etc., can be adopted. As a result of the image recognition, the control unit 16 acquires the position coordinate (XY coordinate) of the tip end portion 3a of the specimen collection device 3 in the X-ray image 41 as the collection position information 45.

As another example of the acquisition method of the collection position information 45, the control unit 16 accepts the specification of the collection position P by an operation input using a pointing device, such as, e.g., a mouse included in the operation unit 19, on the X-ray image 41, for example. In this case, the control unit 16 acquires the position coordinate (XY coordinate) identified on the X-ray image 41 as the collection position information 45.

The collection position information 45 is not limited to a position coordinate (XY coordinate) of the collection position P in the diagnostic image 40. For example, the collection position information 45 is the relative position of the collection position P with respect to the feature point K (see FIG. 8) reflected in the diagnostic image 40. The feature point K includes an anatomical structure, such as, e.g., a blood vessel and a bone, in the diagnostic image 40, an indwelling object M2 (see FIG. 4(C)), such as, e.g., a marker M1 (see FIG. 4(A)) and a stent. As for the anatomical structure, for example as shown in FIG. 8, in the case of a diagnostic image 40 in which the blood vessel branches from the middle, the branch point of the blood vessel can be the feature point K. The feature point K of the anatomical structure is preferably a part which moves almost in unison with the collection position P when a movement of subject T or an organ movement in the subject T occurs and the variation position of the relative position with respect to the collection position P is small.

Further, the collection position information 45 includes, for example, an anatomical name of a part to which the collection position P of the specimen sample 90 belongs. It is preferable that the anatomical name be a part name which is easily recalled by a doctor, such as, e.g., “adrenal vein” and “adrenal cortex”. A plurality of collection position information 45 may be used together.

The control unit 16 associates, for example, by including the collection position information 45 in the image connection data 44 together with the X-ray image 41 and the analysis result 43. In this case, a data element 44a for storing the collection position information 45 is further added to the image connection data 44.

(Image Synthesis)

In the seventh embodiment, the control unit 16 controls the image processing unit 17 so as to synthesize the plurality of X-ray images 41 captured when specimen samples are collected at a plurality of locations in the subject T based on the collection position information 45. As a result, the X-ray imaging apparatus 1 can output a synthesized image 46 that can identify a plurality of collection positions P.

Specifically, as shown in FIG. 22, a base image 46a is acquired with a wide imaging range such that a plurality of collection positions P can be browsed first. In the adrenal vein collecting, the base image 46a is an image that allows the entire adrenal glands to fall within the field of view, for example.

On the other hand, when blood collecting is performed at a collection position P (any of adrenal veins), a magnified image 46b containing only a specific collection position P in the field of view is acquired with movements of the field position and changes of magnification. In this case, the magnified image 46b corresponds to an enlarged image of a part of the base image 46a. The collection position information 45 is obtained, for example, as the position coordinate (Xa, Ya) of the collection position P in the magnified image 46b.

When the base image 46a and the magnified image 46b are acquired, the control unit 16 calculates, for example, the position coordinate of the image center C1 of the base image 46a and the position coordinate of the image center C2 of the magnified image 46b, acquires the movement amount of the moving mechanism 14 and the top board drive unit 15, and then obtains the relative position coordinate of the image center C2 with respect to the image center C1. With this, the control unit 16 calculates the position coordinate of the collection position P in the base image 46a based on the relative position coordinate of the image center C2 of the magnified image 46b with respect to the image center C1 of the base image 46a and the collection position information 45 (position coordinate of the collection position) in the magnified image 46b.

Based on the calculated position coordinate, the control unit 16 synthesizes the magnified image 46b with the base image 46a, and controls the image processing unit 17 (see FIG. 6) so that the position coordinate (Xa, Ya) of the collection position information 45 can be distinguishably displayed in the base image 46a. When the X-ray image 41 (magnified image 46b) indicating another collection position P (Xb, Yb) is acquired, the control unit 16 similarly synthesizes the magnified image 46b with the base image 46a. As a result, o single synthesized image 46 in which the collection position P of each specimen sample 90 is distinguishably displayed is created.

### Effects of Seventh Embodiment

In the seventh embodiment, in the same manner as in the first example, by associating the diagnostic image 40 (X-ray image 41) capable of identifying the collection position P with the specimen identification information 42, it becomes possible to reduce the management burden of the analysis result 43 and the collection position P of the specimen sample 90 when performing the diagnosis by the specimen sample 90 collected from the subject T.

Further, in the seventh embodiment, as described above, the association means 60 is configured so as to further associate the collection position information 45 with the diagnostic image 40 when the specimen sample 90 is collected. With this, it is possible to grasp the collection position P by the collection position information 45 associated with the diagnostic image 40. Therefore, it becomes possible to effectively reduce the management burden of the analysis result 43 and the collection position P of the specimen sample 90.

Further, in the seventh embodiment, as described above, the association means 60 is configured so as to further associate the collection position information 45 with the specimen identification information 42. With this, it is possible to grasp the collection position P by the collection position information 45 associated with the specimen identification information 42. For this reason, it becomes possible to effectively reduce the management burden of the analysis result 43 and the collection position P of the specimen sample 90.

In the seventh embodiment, as described above, the position coordinate (Xa, Ya, etc.) of the collection position P in the diagnostic image 40 is included as the collection position information 45. Thus, by using the position coordinate, the collection position P in the diagnostic image 40 can be clearly and reliably grasped.

Further, in the seventh embodiment, as described above, as the collection position information 45, the relative position of the collection position P with respect to the feature point K appeared in the diagnostic image 40 is included. With this configuration, it is possible to easily grasp the collection position P in the diagnostic image 40 by the relative position of the collection position P with respect to the feature point K in the subject T. Further, the feature point K in the subject T is used as a reference for collection position P. Therefore, for example, when a doctor compares multiple diagnostic images 40, even when the collection position P is shifted between diagnostic images 40 due to the subject T's own movements or the like, as long as the feature point K moves with the collection position P, the collection position P (relative position) with respect to the feature point K does not shift, which enables an accurate grasping of the collection position P.

Further, in the seventh embodiment, as described above, as the collection position information 45, the anatomical name of the part to which the collection position P of the specimen sample 90 belongs is included. With this configuration, the anatomical name makes it possible to intuitively and promptly understand the collection position P when a doctor, etc., refers to the diagnostic image 40. For this reason, it becomes easy to grasp the collection position P and improve the convenience of the diagnostic image 40 system.

### Eighth Embodiment

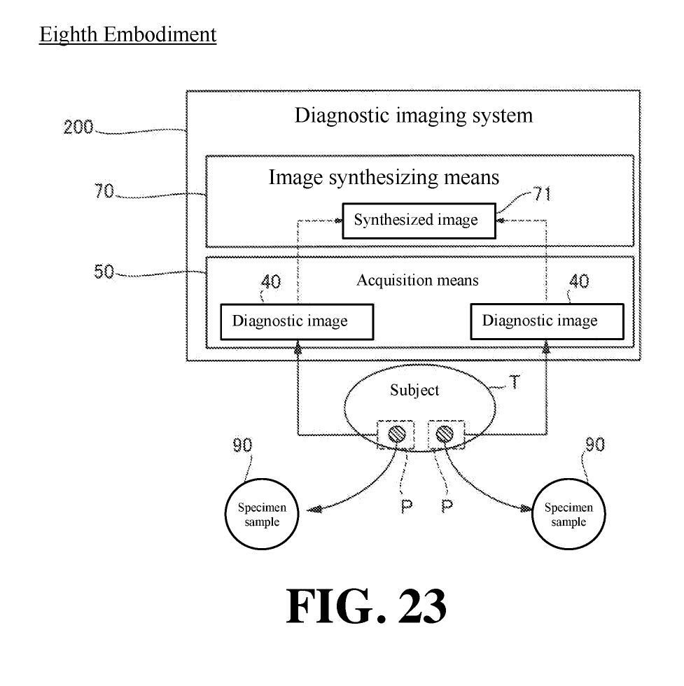

Next, an eighth embodiment will be described with reference to FIG. 23 to FIG. 26.

In the seventh embodiment, the example in which the association between the X-ray image 41 and the specimen identification information 42 is performed and the synthesized image 46 is generated is described. However, in the eighth embodiment, an example of a diagnostic imaging system that generates a synthesized image 46 without performing association will be described.

The diagnostic imaging system 200 according to the eighth embodiment is provided with an acquisition means 50 for acquiring a diagnostic image 40 capable of identifying the collection position P of the specimen sample 90 for each of a plurality of different collection positions P and an image synthesizing means 70 for synthesizing a plurality of diagnostic images 40 to generate a synthesized image 71.

The acquisition means 50 acquires a diagnostic image 40 capable of identifying each collection position P individually when specimen samples 90 are collected separately from a plurality of parts of the subject T.

In the same manner as in the first embodiment, the acquisition means 50 may acquire the diagnostic image 40 of the subject T generated by the image generation apparatus 51 via a transmission medium such as a network or a recording medium, or may acquire the diagnostic image 40 by generating the diagnostic image 40 of the subject T. The diagnostic image 40 is the same as in the first embodiment, and may be any of an X-ray image, a CT image, an Mill image, an ultrasonic image, a nuclear medicine image, and an optical image, or a combination of these images. The diagnostic image 40 may be any of a still image and a moving image.

The image synthesizing means 70 synthesizes a plurality of diagnostic images 40 obtained by the acquisition means 50 by image processing. The image synthesizing means 70 may be configured by an image processing apparatus or the like for synthesizing a plurality of diagnostic images 40. The acquisition means 50 and the image synthesizing means 70 may be configured by an image generation apparatus 51 capable of generating a diagnostic image 40 and performing image processing. The synthesized image 71 may be any one a two-dimensional image and a three-dimensional image. The synthesized image 71 may be, for example, a form of synthesizing a two-dimensional image obtained by enlarging the collection position P on a base three-dimensional image.

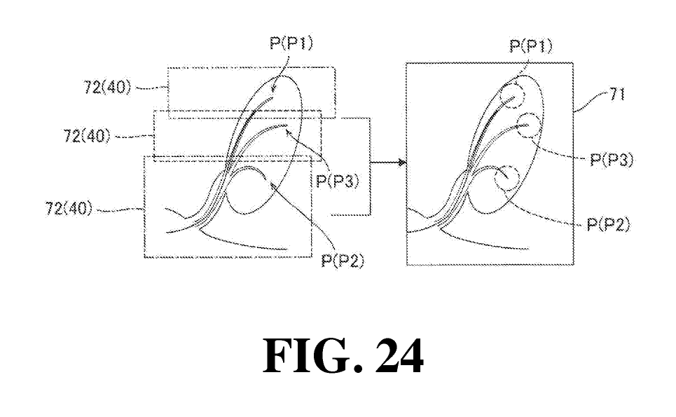

As shown in FIG. 24 to FIG. 26, the image synthesizing means 70 collects images of regions including the collection position P in each diagnostic image 40 to generate a single synthesized image 71.

FIG. 24 shows an example in which a plurality of images 72 obtained by dividing the entire organ (adrenal glands in the example of FIG. 24) to be an examination target (specimen collection target) into a plurality of regions and synthesizing the images 72 by the image synthesizing means 70 to generate a single synthesized image 71 showing the whole of the organ. The image 72 to be synthesized is not necessarily the entirety of the diagnostic image 40 as long as it includes the image part of the region including the collection position P. Also, as long as images including the respective collection positions P are synthesized in the synthesized image 71, the synthesized image 71 may partially include an image in which the collection position P is not reflected. FIG. 24 shows an example in which a plurality of (three) collection positions P1 to P3 are identifiably displayed in a single synthesized image 71 by synthesis.

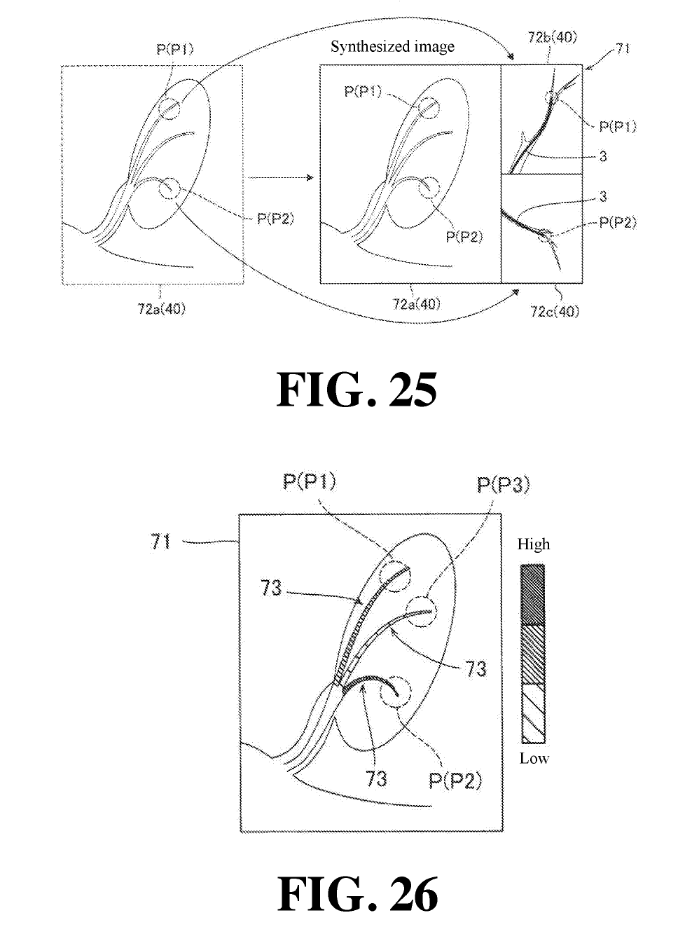

FIG. 25 shows an example in which a single synthesized image 71 is generated by arranging images 72 of regions including the collection position P. Specifically, in FIG. 25, an example is shown in which an image 72a showing the whole of the organ to be examined including the collection positions P1 and P2, an image 72b which enlarges the first point collecting point P1, and an image 72c which enlarges the second point collection position P2 arranged side by side to form a single synthesized image 71.

Note that the configuration shown in FIG. 22 may be adopted. In the configuration example of FIG. 22, the image synthesizing means 70 aligns the image of the region including the collection position P in another diagnostic image 40 with any of the diagnostic images 40 and superimposes them to generate the synthesized image 71 (synthesized image 46). The generation method of the synthesized image 71 is similar to that in the seventh embodiment, so its description is omitted.

In the configuration example of FIG. 26, the image synthesizing means 70 generate the synthesized image 71 to be displayed visually distinguishably by making display colors of the plurality of collection positions P different from each other. FIG. 26 shows an example in which the collection positions P1 to P3 are displayed so as to be distinguishable in a single synthesized image 71. The collection positions P1 to P3 in FIG. 26 are positions near the distal ends of the separate blood vessels. Therefore, the image synthesizing means 70 displays the image portions 73 of the blood vessels corresponding to the collection positions P1 to P3 with different display colors by image processing. Note that in FIG. 26, the difference in display color is indicated by the difference in shading of hatching. It is preferable that the display color is a color which is easily distinguishable visually from the other image portions 73. For example, in the case of a grayscale X-ray image 41, a color different from a gray scale (achromatic color), such as, e.g., red and blue, is selected.

In the configuration example of FIG. 26, the display color may be given merely to distinguish the collection positions P1 to P3, but information of the analysis result may be displayed according to the display color. For example, the image synthesizing means 70 may generate the synthesized image 71 in which the magnitude of the detected amount (or concentration) of the components to be analyzed is displayed by different display colors based on the analysis result 43 of the specimen samples 90 collected at each of the collection positions P1 to P3.

In FIG. 26, an example is shown in which each of the collection positions P1 to P3 is displayed in a gradation or color-coded manner so that the higher the detection amount (concentration) of the component to be analyzed is, the closer to the first display color (dark hatching) such as red and the lower the detection amount (concentration) of the component to be analyzed is, the closer to the second display color (thin hatching). This makes it possible to visually grasp not only the collection position P but also the outline of the analysis result by simply referring to the synthesized image 71.

Each of the configuration examples shown in FIG. 22 and FIG. 24 to FIG. 26 may be employed alone, or may be employed in combination. For example, in FIG. 25, the image 72a showing the entirety of the organ may be generated by a synthesized image of a plurality of images 72 as shown in FIG. 24.

Further, the configuration described in the eighth embodiment may be combined with the first to seventh embodiments, and the synthesized image 71 as the diagnostic image 40, the specimen identification information 42, the subject information 48, the analysis result 43, and the like may be associated with each other.

### Effects of Eighth Embodiment

In the diagnostic imaging system 200 of the eighth embodiment, as described above, the image synthesizing means 70 configured to synthesize a plurality of diagnostic images 40 to generate a synthesized image 71 is provided. With this, it is possible to comprehensively grasp a plurality of collection positions P by the synthesized image 71 obtained by synthesizing a plurality of diagnostic images 40 capable of identifying each collection position P. As a result, by referring to the synthesized image 71 at the time of the diagnosis, the doctor can easily grasp each of the plurality of collection positions P. Further, when explaining the diagnosis result, it is unnecessary to present individual diagnostic images 40 one by one to a patient or to edit each diagnostic image 40 so that the images are be listed. As a result, it becomes possible to make the doctor's diagnosis work and the explanation work to the patient using the diagnostic images 40 more efficient. Further, since a plurality of collected positions P can be collectively grasped by the synthesized image 71, it is possible to reduce the management burden of the analysis result 43 and the collection position P of the specimen sample 90 when conducting a diagnosis by the specimen sample 90 collected from the subject T.

Further, in the eighth embodiment, as described above, the image synthesizing means 70 is configured to collect images of regions including the collection position P in each diagnostic image 40 so as to generate a single synthesized image 71 (see FIG. 24 and FIG. 25). This makes it possible to comprehensively grasp each collection position P in a single synthesized image 71, so that it is easier to further grasp each collection position P by the diagnostic image 40 at the time of diagnosis or explanation to a patient.

Further, in the eighth embodiment, as described above, the image synthesizing means 70 is configured to generate the synthesized image 71 (see FIG. 22) by superimposing the image of the region including the collection position P in the other diagnostic image 40 into any of the diagnostic images 40 while aligning. As a result, the synthesized image 71 makes it possible to grasp, for example, the entire image of the examination target part, and the arrangement and state of individual collection positions P in the entire image at a glance.

Further, in the eighth embodiment, as described above, the image synthesizing means 70 is configured to generate the synthesized image 71 which displays visually distinguishably by making the display colors of the plurality of collection positions P different. With this configuration, since it becomes possible to distinguish a plurality of collection positions P by color as well as position, it becomes possible to easily identify each collection position P at a glance in a synthesized image 71. As a result, the doctor's diagnostic work using diagnostic images 40 can be made more efficient.

### Modified Embodiment

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the second to seventh embodiments, an example is shown in which the image connection data 44 in the DICOM file format is generated as a single data file in which the X-ray image and the analysis result are connected. However, the present invention is not limited to it. In the present invention, a single data file may be generated in a file format other than the DICOM file format.

In the seventh embodiment, an example is shown in which the synthesized image 46 capable of identifying a plurality of collection positions P is included in the image connection data 44, but the present invention is not limited thereto. In the present invention, separately from the image connection data 44, the synthesized image 46 may be outputted as a general-purpose image format (BMP format, JPEG format, or the like). In that case, the collection position P may be recorded directly as an annotation in the synthesized image 46 so that it can be distinguished and displayed on the synthesized image 46.

### DESCRIPTION OF REFERENCE SYMBOLS

1: X-ray imaging apparatus (acquisition means)
2: specimen analyzing device
3: specimen collection device
4: specimen container
8: server
16, 116, 216, 316: control unit (association means)
33, 133, 333: data processing unit (association means)
40: diagnostic image
41: X-ray image
42: specimen identification information (information which identifies the specimen sample collected from the subject)
42a: collection number (identification information)
42b: time information (identification information)
42c: identification information
43: analysis result
45: collection position information (information which identifies the collection position of the specimen sample)
46: synthesized image
48: subject information (information which identifies the subject)
50: acquisition means
60: association means
70: image synthesizing means
71: synthesized image
90: specimen sample
100, 200: diagnostic imaging system
K: feature point
P, P1 to P3: collection position
T: subject

1. A X-ray imaging system comprising:
an acquisition means configured to acquire a X-ray image of a subject; and
an association means configured to associate the X-ray image capable of identifying a collection position when a specimen sample is collected from the subject among the X-ray images acquired by the acquisition means with information which identifies the specimen sample collected from the subject.

2. (canceled)

3. The X-ray imaging system as recited in claim 1,
wherein the X-ray image includes at least one of a two-dimensional image and a three-dimensional image.

4. The X-ray imaging system as recited in claim 1,
wherein the X-ray image includes at least one of a still image and a moving image.

5. The X-ray imaging system as recited in claim 1,
wherein the X-ray image capable of identifying the collection position includes an image capable of identifying the collection position by a specimen collection device arranged at the collection position of the specimen sample or near the collection position.

6. The X-ray imaging system as recited in claim 5,
wherein the specimen collection device includes a collection tool configured to be introduced in the subject to collect the specimen sample in the subject.

7. The X-ray imaging system as recited in claim 1,
wherein the X-ray image capable of identifying the collection position includes an image capable of identifying the collection position by at least one of a marker introduced in the subject and an indwelling object in the subject.

8. The X-ray imaging system as recited in claim 1,
wherein the information which identifies the specimen sample collected from the subject includes identification information assigned for each specimen sample at the time of collection.

9. The X-ray imaging system as recited in claim 1,
wherein the information which identifies the specimen sample collected from the subject includes identification information to be attached to a specimen container for accommodating a collected specimen sample.

10. The X-ray imaging system as recited in claim 1,
wherein the information which identifies the specimen sample collected from the subject includes identification information received from at least one of a specimen analyzing device for analyzing the specimen sample and a server recording an analysis result of the specimen sample.

11. The X-ray imaging system as recited in claim 1,
wherein the association means further associates information which identifies the subject with each of a plurality of X-ray images associated with the information which identifies the specimen sample collected from the subject.

12. The X-ray imaging system as recited in claim 1,
wherein the association means further associates the information which identifies the collection position of the specimen sample in the X-ray image with the X-ray image when the specimen sample is collected.

13. The X-ray imaging system as recited in claim 1,
wherein the association means further associates the information which identifies the collection position of the specimen sample in the X-ray image with the information which identifies the specimen sample collected from the subject.

14. The X-ray imaging system as recited in claim 12,
wherein the information which identifies the collection position includes a position coordinate of the collection position in the X-ray image.

15. The X-ray imaging system as recited in claim 12,
wherein the information which identifies the collection position includes a relative position of the collection position with respect to a feature point reflected in the diagnostic image.

16. The X-ray imaging system as recited in claim 12,
wherein the information which identifies the collection position includes an anatomical name of a part to which the collection position of the specimen sample belongs.

17. The X-ray imaging system as recited in claim 1,
wherein the association means further associates an analysis result of the specimen sample with the information which identifies the specimen sample collected from the subject.

18. The X-ray imaging system as recited in claim 17,
wherein the analysis result of the specimen sample includes a pathological diagnosis result for the specimen sample.

19. The X-ray imaging system as recited in claim 17,
wherein the analysis result of the specimen sample includes a component analysis result for the specimen sample.

20. A X-ray imaging system comprising:
an acquisition means configured to acquire an X-ray image capable of identifying a collection position of a specimen sample for each of a plurality of different collection positions; and
an image synthesizing means configured to synthesize a plurality of X-ray images to generate a synthesized image.

21. The X-ray imaging system as recited in claim 20,
wherein the image synthesizing means collects images of regions including the collection position in each of the X-ray images to generate a single synthesized image.

22. The X-ray imaging system as recited in claim 20,
wherein the image synthesizing means aligns an image of a region including the collection position in another X-ray image with any of the X-ray images and superimposes them to generate the synthesized image.

23. The X-ray imaging system as recited in claim 20,
wherein the image synthesizing means generates the synthesized image to be displayed visually distinguishably by making display colors of the plurality of collection positions different from each other.
